(19) United States
(12) Patent Application Publication
(10) Pub. No.: US 2014/0257200 A1
(43) Pub. Date: Sep. 11, 2014

(54) SAFETY SYSTEM FOR A NEEDLE RETAINING DEVICE

(71) Applicants: Judith Auerbach, Niederteufen (CH); Joerg Geiger, Gais (CH); Levent Kusogullari, Winterthur (CH); C. Garyen Denning, Lexington, KY (US); Andrew J. Corson, Apex, NC (US); Gretchen Elizabeth Willard, Durham, NC (US); David Schmidt, Nicholasville, KY (US)

(72) Inventors: Judith Auerbach, Niederteufen (CH); Joerg Geiger, Gais (CH); Levent Kusogullari, Winterthur (CH); C. Garyen Denning, Lexington, KY (US); Andrew J. Corson, Apex, NC (US); Gretchen Elizabeth Willard, Durham, NC (US); David Schmidt, Nicholasville, KY (US)

(21) Appl. No.: 14/204,883

(22) Filed: Mar. 11, 2014

Related U.S. Application Data
(60) Provisional application No. 61/776,414, filed on Mar. 11, 2013.
(60) Provisional application No. 61/867,356, filed on Aug. 19, 2013.

(51) Int. Cl. A61B 17/34

(52) U.S. Cl. CPC A61B 17/3496
USPC 604/263

(57) ABSTRACT

Embodiments described herein include a safety system including a needle guard assembly, where the needle guard assembly can be configured for attachment to a needle retaining device having a proximal end, a distal end, where a needle can be fixedly coupled to the distal end. The needle guard assembly can include a first cylinder, where the first cylinder can be attached to the distal end of the needle retaining device. The needle guard assembly can include a second cylinder, where the second cylinder can be selectively movable relative to the first cylinder from a first position, to a second position wherein the needle is fully exposed, to a third position where the needle is fully covered. The needle safety system can include a sterile barrier, where the sterile barrier can be configured to maintain the sterility of the needle until use and a flexible hinge, where the flexible hinge can be associated with the needle guard assembly and can lock the second cylinder in the third position such that the second cylinder cannot be advanced proximally in the third position.

### REFERENCE TO RELATED APPLICATIONS

The present application claims the priority benefit of U.S. provisional patent application Ser. No. 61/776,414, filed Mar. 11, 2013, and U.S. provisional patent application Ser. No. 61/867,356, filed Aug. 19, 2013, and hereby incorporates the same applications herein by reference in their entirety.

### TECHNICAL FIELD

Embodiments of the technology relate, in general, to safety syringes, and in particular to passive safety systems that can be attached to a standard syringe, syringe, glass barrel, or carpule to prevent accidental needle sticks, including the transportation and processing of such safety systems.

### BACKGROUND

Needle stick injuries among medical personnel such as healthcare workers are of a growing concern both in developed countries and in the developing world. More than 20 diseases can be transmitted through accidental needle stick injuries particularly Hepatitis B, Hepatitis C and Ebola, West Nile fever and others.

The reduction or elimination of needle stick injuries has become an important issue. Prevention of spread of disease is an important factor in a safe work place and assists recruitment and retention of healthcare workers. The “Needle Stick Safety Act” in the US and similar initiatives enacted in Canada and abroad are forcing the adoption of safer technology for syringes and needles having built-in safety features. In developing countries the inappropriate reuse of syringes has been high. Focus has now been turned to both prevention of disease transmission to healthcare workers by prevention of accidental needlestick injuries and to patients through non reuse of syringes.

### BRIEF DESCRIPTION OF THE DRAWINGS

The present disclosure will be more readily understood from a detailed description of some example embodiments taken in conjunction with the following figures:

FIG. 1 depicts a side view of an example safety system shown attached to a syringe according to one embodiment.

FIG. 2 depicts a side cross-sectional view of the safety system shown in FIG. 1, taken along A-A.

FIG. 3 depicts a perspective view of the safety system shown in FIG. 1 at a first angle.

FIG. 4 depicts a perspective view of the safety system shown in FIG. 1 shown at a second angle.

FIG. 5 depicts a partial side view of the safety system shown in FIG. 1, where the safety system is shown with a portion of the first cylinder cut away, in an initial position, according to one embodiment.

FIG. 6 depicts a partial side view of the safety system shown in FIG. 1, where the safety system is shown with a portion of the first cylinder cut away, in an armed position, according to one embodiment.

FIG. 7 depicts a partial side view of the safety system shown in FIG. 1, where the safety system is shown with a portion of the first cylinder cut away, in an extended or deployed position, according to one embodiment.

FIG. 8 depicts a perspective view of the safety system shown in FIG. 5, where the safety system is shown with a portion of the first cylinder cut away, in an initial position, according to one embodiment.

FIG. 9 depicts a perspective view of the safety system shown in FIG. 6, where the safety system is shown with a portion of the first cylinder cut away, in an armed position, according to one embodiment.

FIG. 10 depicts a perspective view of the safety system shown in FIG. 7, where the safety system is shown with a portion of the first cylinder cut away, in an extended or deployed position, according to one embodiment.

FIG. 11 depicts a right side view of an alternate embodiment of a safety system, shown with an associated syringe prior to attachment, according to one embodiment.

FIG. 12 depicts a right side cross-sectional view of the safety system shown in FIG. 11.

FIG. 13 depicts an exploded perspective view of the syringe and safety system shown in FIG. 11.

FIG. 14 depicts a right side cross-sectional view of the safety system shown in FIG. 11, where the safety system is shown attached to the syringe, in a pre-use position, with a safety cap in place.

FIG. 15 depicts a right side cross-sectional view of the safety system shown in FIG. 11, where the safety system is shown attached to the syringe, in a pre-use position, with the safety cap removed.

FIG. 16 depicts a right side cross-sectional view of the safety system shown in FIG. 11, where the safety system is shown during use, with a telescoping portion retracted or armed, to further expose a needle.

FIG. 17 depicts a right side cross-sectional view of the safety system shown in FIG. 11, where the safety system is shown deployed with the telescoping portion extended to cover the needle.

FIG. 18 depicts a perspective view of an inner cylinder according to one embodiment.

FIG. 19 depicts a perspective view of a middle cylinder according to one embodiment.

FIG. 20 depicts a perspective view of an outer cylinder accord to one embodiment.

FIG. 21 depicts a right side partial cross-sectional view of the inner cylinder shown in FIG. 18 and the middle cylinder shown in FIG. 19, where the inner cylinder is shown engaged with the middle cylinder in a pre-use position.

FIG. 22 depicts a right side partial cross-sectional view of the inner cylinder shown in FIG. 18 and the middle cylinder shown in FIG. 19, where the inner cylinder is shown engaged with the middle cylinder when the telescoping portion is fully retracted or armed and the needle is fully exposed.

FIG. 23 depicts a right side partial cross-sectional view of the inner cylinder shown in FIG. 18 and the middle cylinder shown in FIG. 19, where the inner cylinder is shown engaged with the middle cylinder when the telescoping portion is substantially extended, but not yet locked or fully deployed.

FIG. 24 depicts a right side partial cross-sectional view of the inner cylinder shown in FIG. 18 and the middle cylinder shown in FIG. 19, where the inner cylinder is shown engaged with the middle cylinder when the telescoping portion is in a fully extended and locked position and the needle is covered.

FIG. 25 depicts a perspective view of an alternate version of a safety system shown attached to a syringe.

FIG. 26 depicts a perspective exploded view of the safety system of FIG. 25.

FIG. 27 depicts a right side cross-sectional view of the safety system of FIG. 25, shown in a pre-use configuration with a cap in place.

FIG. 28 depicts a right side cross-sectional view of the safety system of FIG. 25, shown in a pre-use configuration with the cap removed.

FIG. 29 depicts a right side cross-sectional view of the safety system of FIG. 25, shown during use with a tension spring and a guard in a retracted position.

FIG. 30 depicts a right side cross-sectional view of the safety system of FIG. 25, shown after use with the guard in a locked distal or deployed position.

FIG. 31 depicts a perspective view of an alternate version of a safety system according to one embodiment.

FIG. 32 depicts an exploded view of the safety system of FIG. 31.

FIG. 33 depicts a right side cross-sectional view of the safety system of FIG. 30 shown in a pre-use configuration with a cap in place.

FIG. 34 depicts a right side cross-sectional view of the safety system of FIG. 30 shown in a pre-use configuration with the cap removed.

FIG. 35 depicts a right side cross-sectional view of the safety system of FIG. 30 shown in a retracted position.

FIG. 36 depicts a right side cross-sectional view of the safety system of FIG. 30 shown in a fully deployed position.

FIG. 37 depicts a cross-sectional view of a safety system, according to an alternate embodiment, shown in a deployed position.

FIG. 38 depicts a perspective view of the safety system shown in FIG. 37.

FIG. 39 depicts a cross-sectional view of a safety system according to an alternate embodiment.

FIG. 40 depicts a left side cross-sectional view of a safety system according to an alternate embodiment.

FIG. 41 depicts a perspective view of a safety system, according to an alternate embodiment, shown with a needle guard assembly disengaged from a needle retaining device.

FIG. 42 depicts a perspective view of the safety system shown in FIG. 41, where the needle guard assembly is shown partially engaged with the needle retaining device.

FIG. 43 depicts a perspective view of the safety system shown in FIG. 41, where the needle guard assembly is shown fully engaged with the needle retaining device.

FIG. 44 depicts a cross-sectional view of a safety system according to an alternate embodiment.

FIG. 45 depicts a cross-sectional view of a safety system according to an alternate embodiment.

FIG. 46 depicts partial cross-sectional view of a safety system according to an alternate embodiment.

FIG. 47 depicts a partial cross-sectional view of a safety system according to an alternate embodiment.

FIG. 48 depicts a partial cross-sectional view of a safety system, according to one embodiment, shown in a first position.

FIG. 49 depicts a partial cross-sectional view of the safety system shown in FIG. 48, where the safety system is shown in a second position.

FIG. 50 depicts a cross-sectional view of a second cylinder associated with the safety system shown in FIG. 48.

FIG. 51 depicts a cross-sectional view of a safety system according to an alternate embodiment.

FIG. 52 depicts a partial cross-sectional view of a safety system according to an alternate embodiment.

FIG. 53 depicts a right side cross-sectional view of a safety system according to an alternate embodiment.

FIG. 54 depicts a cross-sectional view of a safety system according to an alternate embodiment.

FIG. 55 depicts a partial cross-sectional view of a safety system according to an alternate embodiment.

FIG. 56 depicts a partial cross-sectional view of a safety system and an associated assembly tool according to an alternate embodiment.

FIG. 57 depicts a partial cross-sectional view of a safety system and an associated assembly tool according to an alternate embodiment.

FIG. 58 depicts a partial cross-sectional view of a transportation or storage system for a safety system according to one embodiment.

FIG. 59 depicts a partial top view of a nest associated with the transportation or storage system shown in FIG. 58.

### SUMMARY

Embodiments described herein include a safety system comprising a needle guard assembly, where the needle guard assembly can be configured for attachment to a needle retaining device having a proximal end, a distal end, where a needle can be fixedly coupled to the distal end. The needle guard assembly can include a first cylinder, where the first cylinder can be attached to the distal end of the needle retaining device. The needle guard assembly can include a second cylinder, where the second cylinder can be selectively movable relative to the first cylinder from a first position, to a second position wherein the needle is fully exposed, to a third position where the needle is fully covered. The needle safety system can include a sterile barrier, where the sterile barrier can be configured to maintain the sterility of the needle until use and a flexible hinge, where the flexible hinge can be associated with the needle guard assembly and can lock the second cylinder in the third position such that the second cylinder cannot be advanced proximally in the third position.

Embodiments described herein can include a safety system comprising a needle guard assembly, where the needle guard assembly can be configured for attachment to a needle retaining device having a proximal end, a distal end, and a needle, where the needle can be fixedly coupled to the distal end. The needle guard assembly can include a telescoping region having a first cylinder, where the first cylinder can be attached to the distal end of the needle retaining device, a second cylinder, where the second cylinder can be selectively movable relative to the first cylinder, a third cylinder, where the third cylinder can be selectively movable relative to the second cylinder, where the telescoping region can include a first position, a second position where the needle is fully exposed, and a third position where the needle is fully covered. The safety system can include a flexible hinge, where the flexible hinge can be associated with the needle guard assembly and can locks the second cylinder in the third position such that the second cylinder cannot be advanced proximally in the third position.

Embodiments described herein can include a safety system comprising a needle guard assembly, where the needle guard assembly can be configured for attachment to a needle retaining device having a proximal end, a distal end, and a needle, where the needle can be fixedly coupled to the distal end. The needle guard assembly can include a first cylinder, where the first cylinder can be attached to the distal end of the needle retaining device, a second cylinder, where the second cylinder can be selectively movable relative to the coupling cylinder from a first position where the needle is fully covered, to a second position where the needle is fully exposed, to a third position where the needle is fully covered. The safety system can include a tension spring, where the tension spring can couple the second cylinder with the first cylinder. The safety system can include a flexible hinge, where the flexible hinge can be associated with the needle guard assembly and can locks the second cylinder in the third position such that the second cylinder cannot be advanced proximally in the third position.

Embodiments described herein can include a safety system comprising a needle guard assembly, where the needle guard assembly can be configured for attachment to a needle retaining device having a proximal end, a distal end, and a needle, where the needle can be fixedly coupled to the distal end. The needle guard assembly can include a first cylinder, where the first cylinder can be attached to the distal end of the needle retaining device, a second cylinder, where the second cylinder can be selectively movable relative to the first cylinder from a first position, to a second position where the needle is fully exposed, to a third position where the needle is fully covered. The second cylinder can include a first annular portion that can be positioned within the first cylinder and a second annular portion that can be positioned outside of the first cylinder. The safety system can include a spring, where the spring can be positioned between the first cylinder and the second annular portion of the second cylinder. The safety system can include a flexible hinge, where the flexible hinge can be associated with the needle guard assembly and can locks the second cylinder in the third position such that the second cylinder cannot be advanced proximally in the third position.

A system for transporting a safety system can include a needle retaining device, the needle retaining device having a proximal end and a distal end, where a needle can be fixedly coupled to the distal end of the needle retaining device. The system can include a needle guard assembly having a proximal end and a distal end, the needle guard assembly can be coupled to the distal end of the needle retaining device, where the needle guard assembly can include a coupling cylinder attached to the distal end of the needle retaining device and a movable guard that can be selectively movable relative to the coupling cylinder. The system can include a tub, a tray, where the tray can be positioned within the tub, where the distal end of the needle guard assembly can be retained by the tray. The system can include a nest, where the needle retaining device can be retained by the nest and can be substantially restricted from movement by one or a plurality of flexible elements associated with the nest.

### DETAILED DESCRIPTION

Various non-limiting embodiments of the present disclosure will now be described to provide an overall understanding of the principles of the structure, function, mechanisms, functionality and use of the safety systems and processes disclosed herein. Various non-limiting embodiments of process steps and transportation systems related to the safety systems are disclosed. Various non-liming embodiments of barrier systems associated with safety systems are disclosed. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Example embodiments described herein can be used to prevent accidental needle sticks. Example embodiments can include a safety system that can be attached to a standard off-the-shelf syringe, syringe, glass barrel, or pre-filled cartridge. Embodiments can be integrated into a standard prefilled syringe by, for example, a glass company. Embodiments can include automatic, passive needle stick safety protection that can comply with US and EU guidelines for needle stick safety prevention. Embodiments for the safety system can use current stability packaging materials of a validated pre-filled safety system or modified packaging material to fit existing standards. Embodiments can provide a needle stick safety solution that may be implemented with lower total cost than, for example, bolt-on safety solutions.

Example embodiments described herein can include any suitable components or elements that can be configured to create or establish a sterile barrier system for a pre-filled syringe, or the like. Versions of such a system can protect the stability of pre-filled drugs, medicament, or other chemicals stored within a pre-filled syringe, or the like. Such sterile barrier systems can maintain the sterility of the drug, needle, or any other component or element. Example embodiments can include using existing glass-syringe needle caps that have already been validated as part of a sterile barrier system, or can include unique enclosure or barrier system elements as described in more detail herein, or any combination thereof. Unique enclosure or barrier system elements can include design features that can occlude the needle tip of a pre-filled syringe for the entire shelf life of the device, pre-filled syringe, or medicament contained therein. Such enclosures or barrier systems can be configured to prevent the loss of drugs or drug stability and can include features that can allow the needle to remain sterile for the shelf life of the drug, the pre-filled syringe, or the device. Such systems can be configured to be effective when the device or pre-filled syringe is removed from, or is otherwise unassociated with, sterile barrier packaging.

Example embodiments described herein can be used with a standard needle, a standard glass hub, a standard cone, a standard needle shield, or a standard fixture to the glass hub of a syringe or pre-filled cartridge. Embodiments can include a one-step assembly of the safety system by a manufacture, where, for example, a manufacturer can pre-assemble the safety system. Embodiments can fit in standard three inch or four inch tubs and can fit in 10×16 or 10×10 tubs, for example.

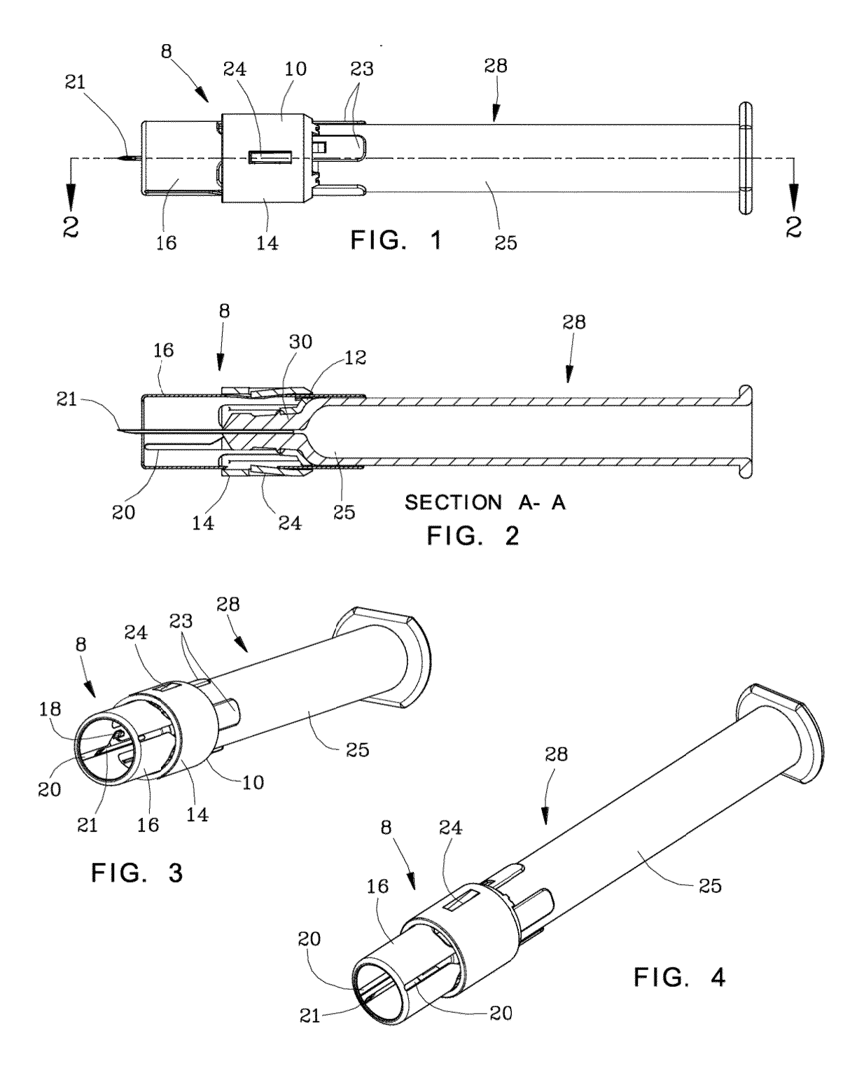

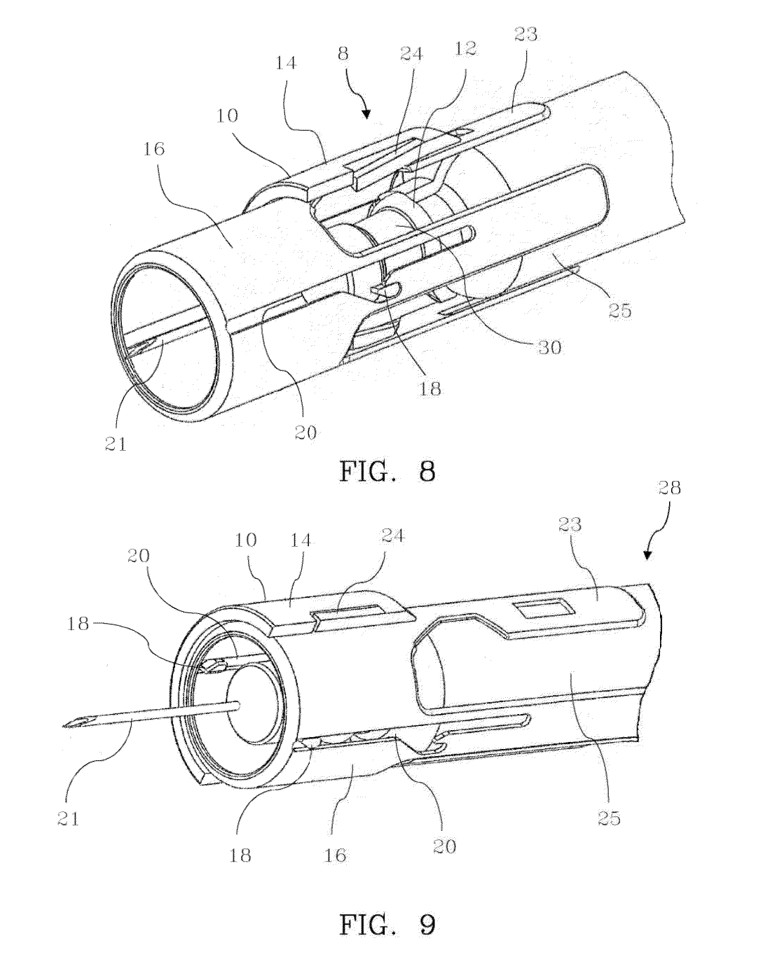

Referring now to FIGS. 1-10, an automatic non-reusable needle guard 8 can include a first cylinder 10 that can be attached to a needle retaining device 28, such as a standard syringe, syringe, carpule, or pre-filled cartridge that can retain a needle 21. The first cylinder 10 can have an inner portion 12 (FIG. 8) and an outer portion 14 (FIG. 8). In an example embodiment, the inner portion 12 can be coupled with a neck 30 of the standard needle retaining device 28 with a snap fit, friction fit, gluing, or any other suitable coupling. It will be appreciated that the coupling can be associated with a sterile barrier that can maintain the sterility of the needle 21 and a medicament or drug contained within a pre-filled syringe. It will be appreciated that the needle guard 8 can have any suitable shape, size, or configuration.

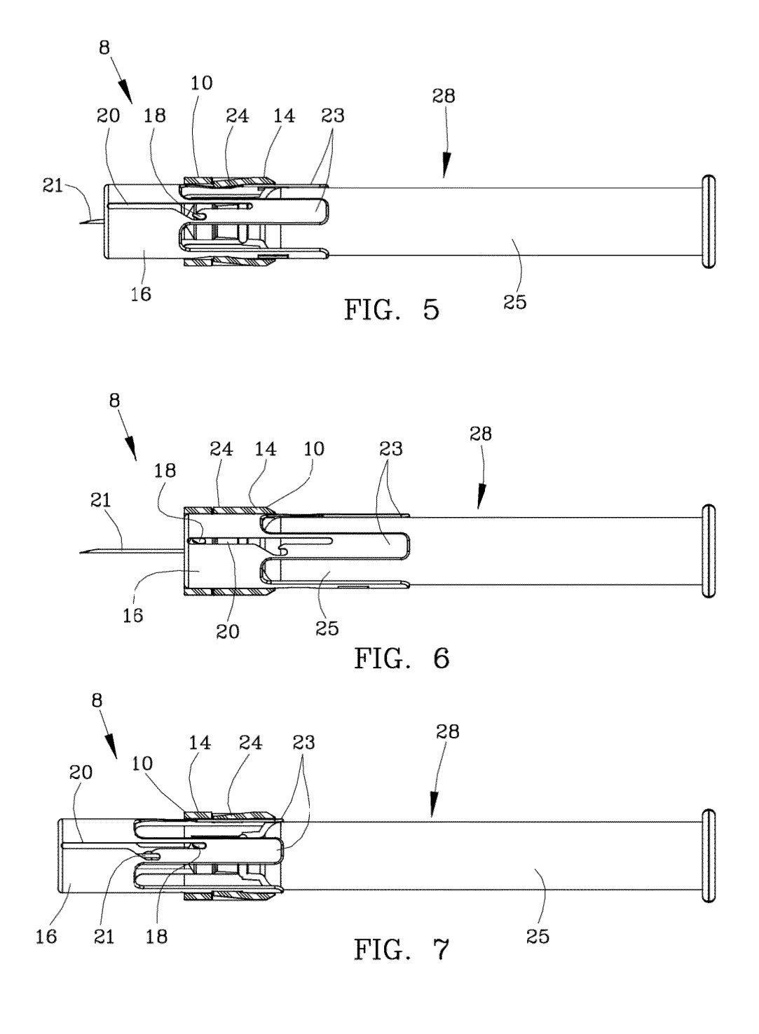

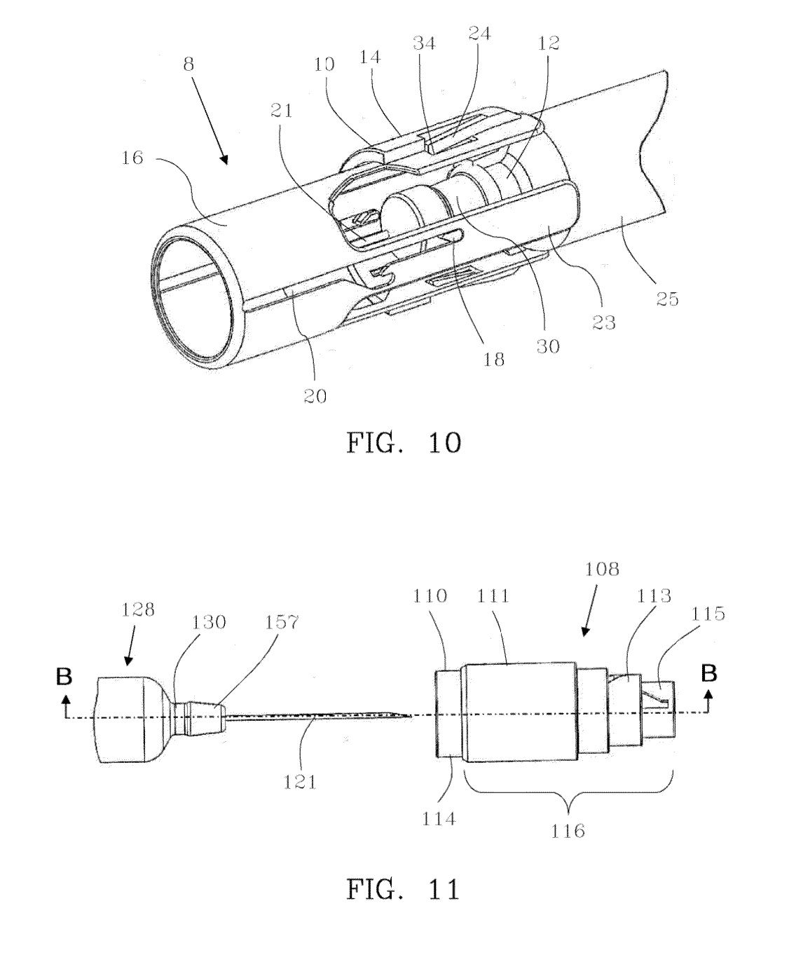

The automatic non-reusable needle guard 8 can include a second cylinder 16 that can be operable with the first cylinder 10 for movement of the second cylinder 16 from an initial retractable position (FIGS. 5 and 8) to a released or armed position (FIGS. 6 and 9) and further to an extended irreversible position (FIGS. 7 and 10). The second cylinder 16 can include a plurality of flanges 23 that can extend axially along a portion of the needle retaining device 28. The flanges may help stabilize the needle guard 8 relative to the needle retaining device 28. In an alternate embodiment, the needle guard 8 may not have any flanges where, for example, no portion of the needle guard 8 can project proximally over the body 25 of the needle retaining device 28. Such an alternate configuration may facilitate visualization of the contents within the needle retaining device 28. The needle guard 8 can be configured such that the needle 21 can project distally past the needle guard 8 in a pre-use first position, where such a configuration may help clinicians position an injection or the like.

The mechanism that can provide or facilitate movement of the second cylinder 16 relative to the first cylinder 10 can incorporate a first track 20, as shown in more detail in FIGS. 5-10, or a plurality of tracks, where the one or a plurality of tracks can have any suitable shape, such as a J-shape or a generally linear shape having a lateral track projection or detent. The outer portion 14 of the first cylinder 10 can incorporate a first protruding member 18 (FIGS. 3 and 5-10) which can be formed on the outer portion 14 of the first cylinder 10 and can be positioned in the first track 20 such that the first track can guide the second cylinder from an initial usable position to a released position and further to an extended irreversible position. A spring (not shown) can be disposed between the second cylinder 16 and the inner portion 12 of the first cylinder 10 and can urge the second cylinder 16 distally away from the first cylinder 10 such that the inner cylinder 16 can be extended to the extended irreversible position. It will be appreciated that the first track 20 is shown by way of example only, where the first track 20 may have any suitable shape and configuration. It will be appreciated that the one or a plurality of tracks can include J-shapes, G-shapes, linear tracks, cutouts in a cylinder, shape components that can form a channel, guiding notches, molded channels in plastic components, slopes, guiding features, or the like. It will be appreciated that one or a plurality of tracks may be incorporated to achieve a similar operation and it will be appreciated that the track and/or projection can be associated with any suitable component.

Example embodiments of the automatic non-reusable needle guard 8 can also include a locking mechanism (FIGS. 8-10) that can prevent retraction of the second cylinder 16, relative to the first cylinder 10, after the second cylinder 16 is fully extended in the extended irreversible position. A flexible hinge 24 can be associated with the outer portion 14 of the first cylinder 10 where the flexible hinge 24 can be biased radially inward. In the initial position, the flexible hinge 24 can be biased inwardly in its resting state (FIG. 8) in a first position. In the released or armed position (FIG. 9) the flexible hinge 24 can be flexed radially outwardly into a second position as the second cylinder 16 is rotated and axially translated relative to the first cylinder 10. In the extended irreversible position (FIG. 10), the flexible hinge 24 can flex radially inward into a third position such that the flexible hinge 24 can engage a slot 34 defined by the second cylinder 16. In one embodiment, when the flexible hinge 24 has flexed radially inward into the third position and has engaged the slot 34, the second cylinder 16 cannot be moved proximally to expose the distal tip of the needle due to the abutment between the flexible hinge 24 and the slot 34. Such a configuration may prevent accidental needle sticks after use of the needle guard 8. Such a configuration can prevent re-use of the safety system and associated pre-filled syringe after use, such that the assembly can be configured for single use applications.

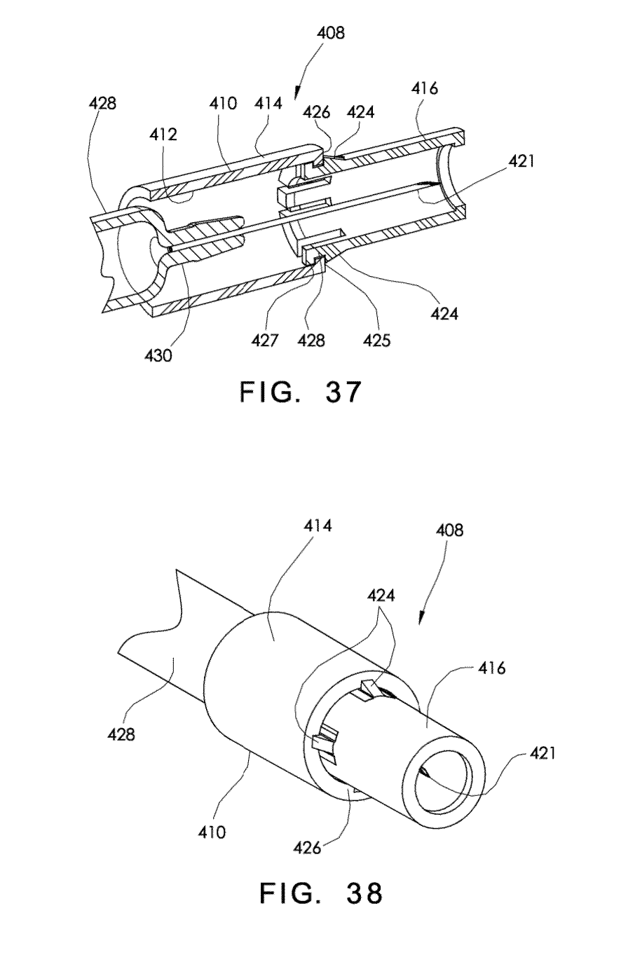

Referring now to FIGS. 37 and 38, an alternate embodiment of an automatic non-reusable needle guard 408 can include a first cylinder 410 that can be attached to a needle retaining device 428, such as a standard syringe, syringe, carpule, or pre-filled cartridge that can retain a needle 421. The first cylinder 410 can have an inner portion 412 and an outer portion 414. In an example embodiment, the inner portion 412 can be coupled with a neck 430 of the standard needle retaining device 428 with a snap fit, friction fit, or any other suitable coupling. It will be appreciated that the coupling can be associated with a sterile barrier that can maintain the sterility of the needle 421 and a medicament or drug contained within a pre-filled syringe. It will be appreciated that the needle guard 408 can have any suitable shape, size, or configuration.

The automatic non-reusable needle guard 408 can include a second cylinder 416, or a plurality of cylinders, that can be operable with the first cylinder 410 for movement of the second cylinder 416 from a retractable position (not shown) to an extended irreversible position (FIGS. 37 and 38). It will be appreciated that the automatic non-reusable needle guard can function in the same manner as needle guard 8 described with reference to FIGS. 1-10. The needle guard 408 can include a one or a plurality of flexible hinges 424 or other suitable locking features that can be positioned on the outer surface of the second cylinder 416. The one or a plurality of flexible hinges 424, where the hinges can be biased radially outward. Referring to FIG. 37, the one or a plurality of flexible hinges 424 can be urged radially (not shown) inward by the inner portion 412 of the first cylinder 410 when the needle guard is in a retracted position and then locked out, as illustrated, in a deployed or distal-most position. Referring to FIG. 38, when the needle guard 408 is in the deployed or extended irreversible position, the one or a plurality of flexible hinges 424 can project radially outward such that they assume a relaxed deflected position. When projected radially outward, the one or a plurality of living hinges 424 can prevent the second cylinder 416 from being advanced proximally relative to the first cylinder 410 due to the abutment between the proximal region 425 of the one or a plurality of flexible hinges 424 and the distal end 426 of the inner cylinder 410. When in the initial usable position or a released or position, these locking features can be pressed inward by contact with the inner surface 412 of the first cylinder 410, which can allow for relative proximal or distal longitudinal movement of the first cylinder 410 and second cylinder 416. The needle guard 408 can include a stop 427 that together with the one or a plurality of flexible hinges 424 can define a channel 428 that can prevent both proximal and distal movement in the deployed position. It will be appreciated that the one or a plurality of flexible hinges 424 can include inwardly biased elements, outwardly biased elements, locking members, flexible fingers, ribs, or the like. In an alternate embodiment, the flexible hinges can be in a relaxed state prior to the irreversible deployment of the guard, where the flexible hinges can be deflected radially inward just prior to locking out the guard. In the manner, the safety system may have a longer shelf life as the flexible members may not be retained in a stressed position while not in use.

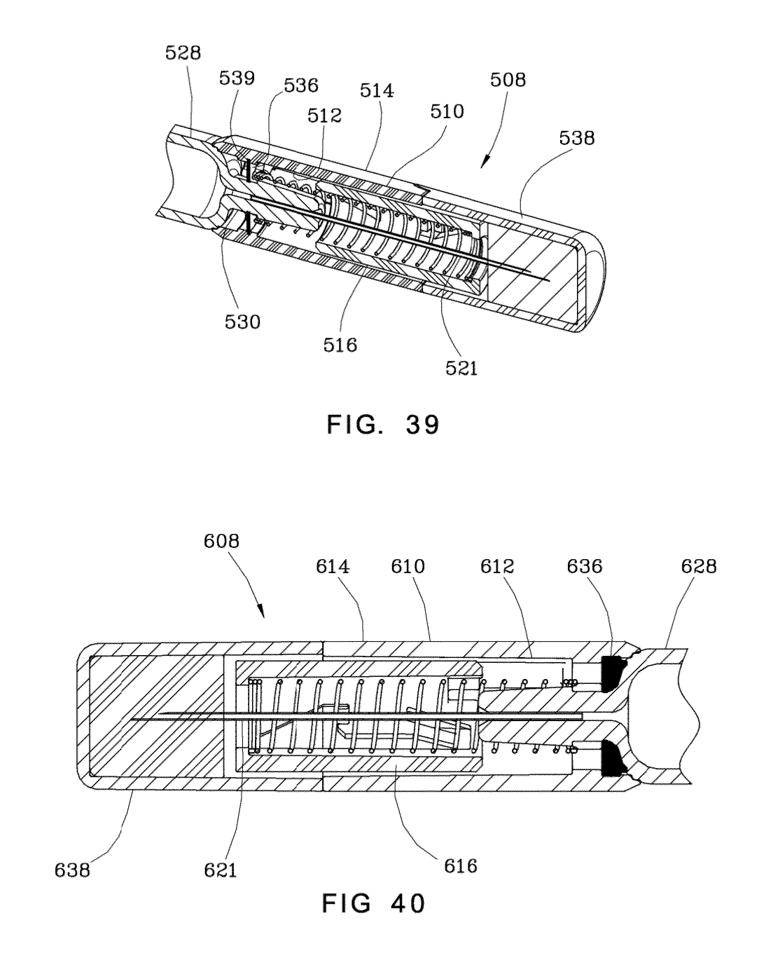

Referring now to FIG. 39, an automatic non-reusable needle guard 508 can include a first cylinder 510 that can be attached to a needle retaining device 528, such as a standard syringe, syringe, carpule, or pre-filled cartridge that can retain a needle 521. The first cylinder 510 can have an inner portion 512 and an outer portion 514. In an example embodiment, the inner portion 512 can be coupled with a neck 530 of the standard needle retaining device 528 with a snap fit, friction fit, or any other suitable coupling. It will be appreciated that the coupling can be associated with a sterile barrier that can maintain the sterility of the needle 521 and a medicament or drug contained within a pre-filled syringe. It will be appreciated that the needle guard 508 can have any suitable shape, size, or configuration.

The automatic non-reusable needle guard 508 can include a second cylinder 516 that can be operable with the first cylinder 510 for movement of the second cylinder 516 from an initial retractable position, to a released or armed position, and further to an extended irreversible position. The needle guard 508 can include a sealed co-molded coupling 536 that can be retained between the first cylinder 510 and the neck 530 of the standard needle retaining device 528. When combined with a pre-use cap 538, which can be sealed to the distal end of the first cylinder 510, the sealed coupling 536 can preserve the sterility of the needle 521, needle hub, and internal surfaces of the needle guard 508. In one embodiment of the sealed coupling 536, TPE, rubber, or other elastomeric features can be co-molded onto a proximal flange 539 of the first cylinder 510. In such an embodiment, the sterile barrier between the first cylinder 510 and the neck 530 of the standard needle retaining device 528 can be maintained by the compression of the sealed coupling 536 against the neck 530. The sterile barrier between the sealed coupling 536 and the first cylinder 510 can be maintained by the co-molded adhesion between the two components, for example. It will be appreciated that the example configurations, cylinders, and associated tracks can be used with a two cylinder design, a three cylinder design, or any suitable design.

Referring now to FIG. 40, an automatic non-reusable needle guard 608 can include a first cylinder 610 that can be attached to a needle retaining device 628, such as a standard syringe, syringe, carpule, or pre-filled cartridge that can retain a needle 621. The first cylinder 610 can have an inner portion 612 and an outer portion 614. In an example embodiment, the inner portion 612 can be coupled with a neck 630 of the standard needle retaining device 628 with a snap fit, friction fit, or any other suitable coupling. It will be appreciated that the coupling can be associated with a sterile barrier that can maintain the sterility of the needle 621 and a medicament or drug contained within a pre-filled syringe. It will be appreciated that the needle guard 608 can have any suitable shape, size, or configuration. It will be appreciated that the elements or features associated with the needle guard 608 that can couple to the needle retaining device 628 can be integral with the needle guard, integral with the first cylinder, co-molded with the needle guard, or otherwise configured.

The automatic non-reusable needle guard 608 can include a second cylinder 616 that can be operable with the first cylinder 610 for movement of the second cylinder 616 from an initial retractable position, to a released or armed position, and further to an extended irreversible position. An alternate embodiment of a sealed coupling 636 can include a separate rubber, TPE, or elastomeric seal that can be used in place of a co-molded sealed coupling 536 (FIG. 39). The sealed coupling 636 can be retained or held within the interior region of the first cylinder 610, or on any intermediate part not shown herein, by mechanical retention features such as, for example, a press fit interference fit between the sealed coupling 636 and the inner portion 612 of the inner cylinder 610, which can restrict the proximal or distal movement of the sealed coupling 636. In such an embodiment, the sterile barrier between the sealed coupling 636 and the first cylinder 610, and the sterile barrier between the sealed coupling 636 and the neck 630 of the standard needle retaining device 628, can both be maintained by the compression of the sealed coupling 636 between such components. In an alternate embodiment, the compression of the sealed coupling 636 can preclude the need for additional retention features in the interior region of the first cylinder 610. The needle guard can also include an end cap 638 that can facilitate a sterile barrier during storage. It will be appreciated that the example configurations, cylinders, and associated tracks can be used with a two cylinder design, a three cylinder design, or any suitable design.

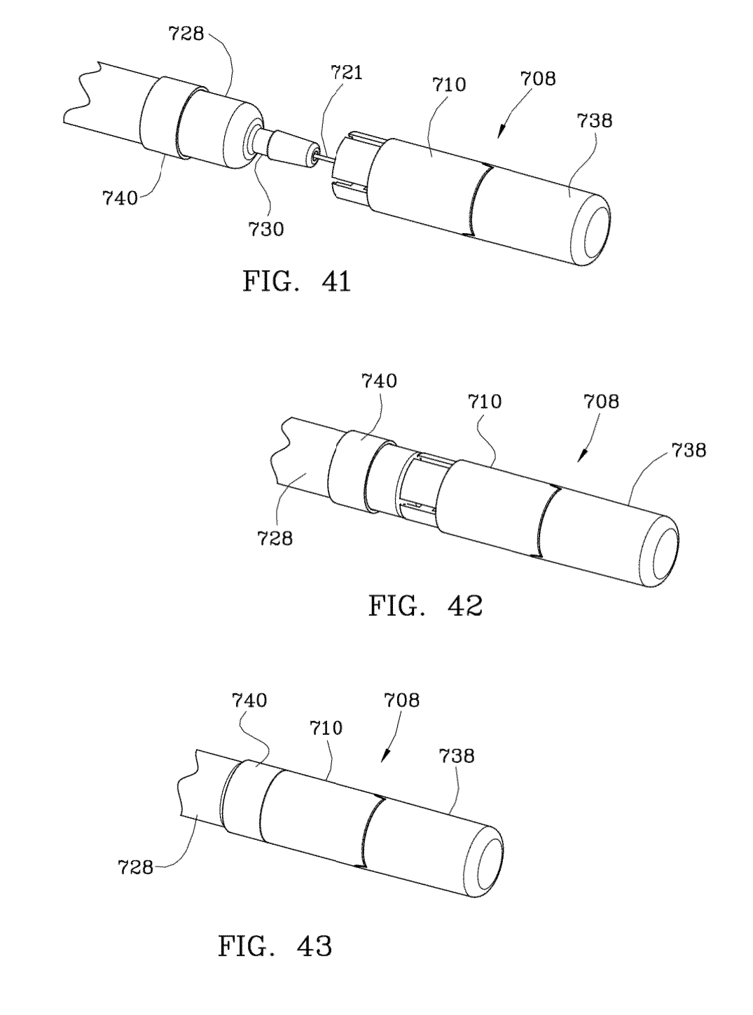

Referring now to FIGS. 41-43, an automatic non-reusable needle guard 708 can include a first cylinder 710 that can be attached to a needle retaining device 728, such as a standard syringe, syringe, carpule, or pre-filled cartridge that can retain a needle 721. The first cylinder 710 can be coupled with a neck 730 of the standard needle retaining device 728 with any suitable coupling. It will be appreciated that the coupling can be associated with a sterile barrier that can maintain the sterility of the needle 721 and a medicament or drug contained within a pre-filled syringe in combination with an end cap 738. It will be appreciated that the needle guard 708 can have any suitable shape, size, or configuration.

The automatic non-reusable needle guard 708 can include a second cylinder (not shown) that can be operable with the first cylinder 710 for movement of the second cylinder from an initial retractable position, to a released or armed position, and further to an extended irreversible position. An alternate embodiment of a sealed coupling is shown in FIGS. 41-43, where a robust coupling and a sterile barrier between the first cylinder 710 and the neck 730 of the standard needle retaining device 728 can be maintained by the compression of a proximal first-cylinder flange 740 on the neck 730. In such an arrangement, the sterile barrier could be maintained either by a single contact sealing surface, or a multiplicity of such features which are not illustrated. Referring to FIG. 41, the needle guard 708 and flange 740 are shown disengaged. Referring to FIG. 41, the inner cylinder 710 of the needle guard 708 can be engaged with the retaining device 728, where the flange 740 has not yet been engaged. In one embodiment, the needle guard 708 can be selectively coupled with the neck 730 of the retaining device 728. Referring to FIG. 43, the flange 740 can be positioned over the proximal end of the inner cylinder 710 being mounted from the bottom or the top such that the needle guard 708 is fixedly engaged with the retaining device 728 and a sterile barrier is created within the needle guard 708. The flange 740 can be configured such that it has a smaller inner diameter than the outer diameter of the proximal end of the inner cylinder 710 such that the proximal end of the inner cylinder 710 can be pressed laterally inward to engage the neck 730. The same functionality can be realized in any suitable manner where, for example, an inner portion or cylinder (not shown) can operate as a coupling and an outer portion or outer cylinder (not shown) can function as a flange to secure the inner portion to the neck of the syringe.

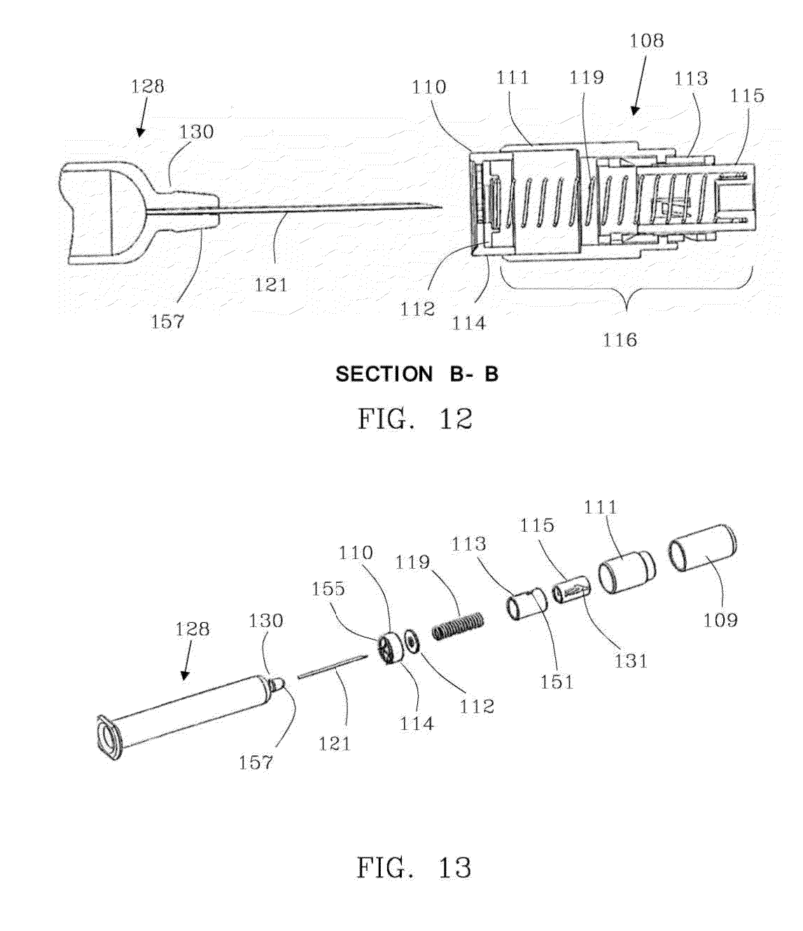

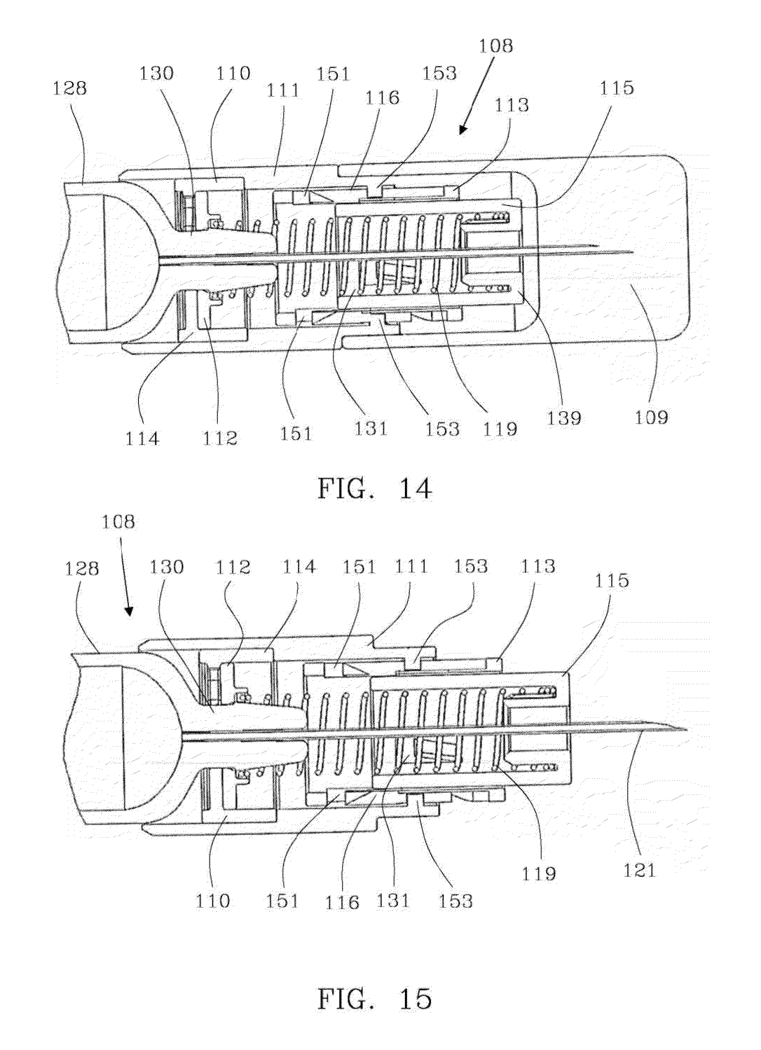

Referring to FIGS. 11-24, an automatic non-reusable needle guard 108 can include a coupling cylinder 110 that can be attached to a needle retaining device 128, such as a standard syringe or pre-filled cartridge that can retain a needle 121. The coupling cylinder 110 can have a seal 112 and a neck portion 114 (FIG. 12). In an example embodiment, the neck portion 114 can be coupled with the neck 130 of the standard needle retaining device 128 with a snap fit, friction fit, form fit, gluing, or any other suitable coupling. It will be appreciated that needle guard 108 can have any suitable shape, size, or configuration. It will be appreciated that the coupling can be associated with a sterile barrier that can maintain the sterility of the needle 121 and a medicament or drug contained within a pre-filled syringe. The needle guard can include a pre-use cap 109 (FIGS. 13 and 14) during shipping or storage that can be removed just prior to use. The pre-use cap 109, as described in more detail herein, can be configured to create a sterile barrier by including a tortuous path or other suitable configuration, design, or construction.

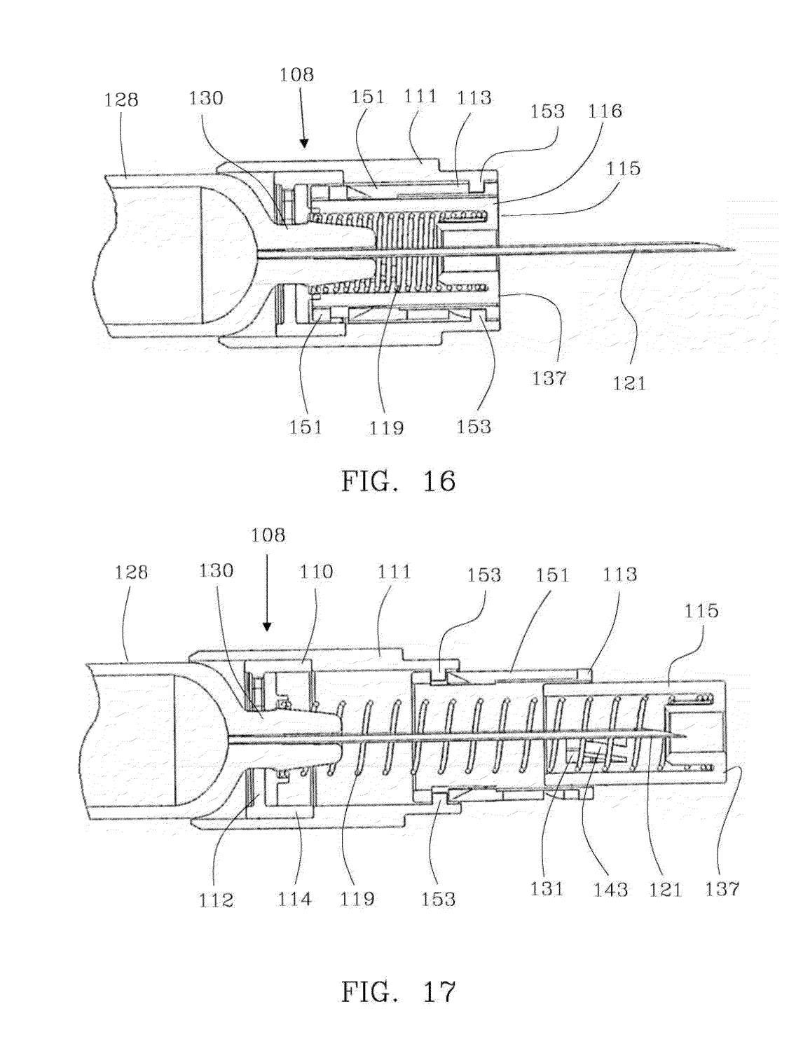

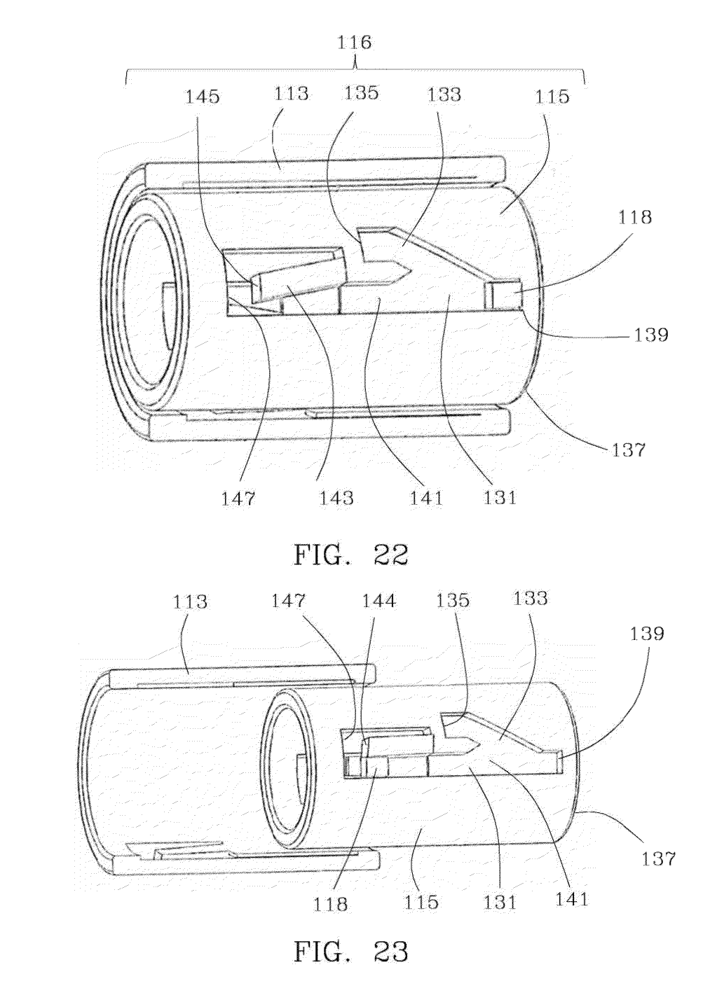

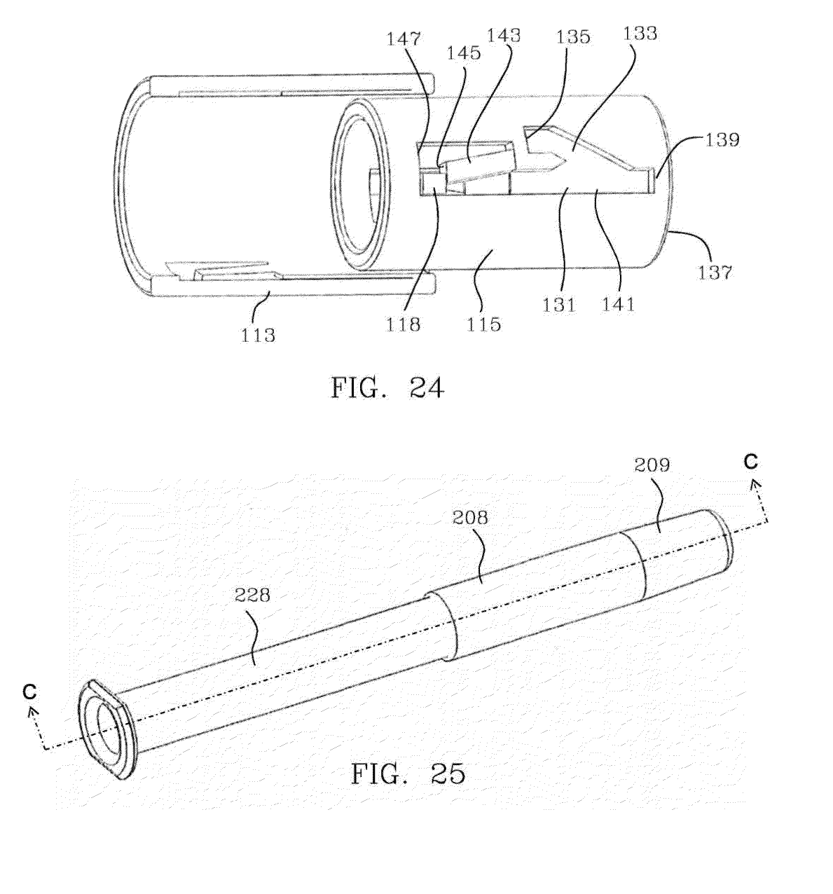

The automatic non-reusable needle guard 108 can include a telescoping region 116, where the telescoping region 116 can include an outer cylinder 111, a middle cylinder 113, and an inner cylinder 115, where the telescoping region can be biased distally with a spring 119. The telescoping region 116 can be configured to transition from an initial retractable position (FIGS. 14, 15, 21), to a retracted position (FIGS. 16 and 22), to an extended and locked position (FIGS. 17 and 24). It will be appreciated that the telescoping region 116 can include any suitable number of telescoping components, including telescoping cylinders, in accordance with systems described herein. It will be appreciated that the telescoping components, including the telescoping cylinders 111, 113, 115, can include any suitable telescoping mechanism or coupling. It will be appreciated that the cylinders can be configured to move concomitantly, where the cylinders can move simultaneously or at the same time such as in a generally distal direction. In an alternate embodiment, the cylinders can move in series where a first cylinder can move or advance before a second cylinder moves or advances. It will be appreciated that cylinders are described by way of example, where any shape such as boxes or polygons is contemplated. Cylindrical or cylinder can mean substantially or generally cylindrical in nature.

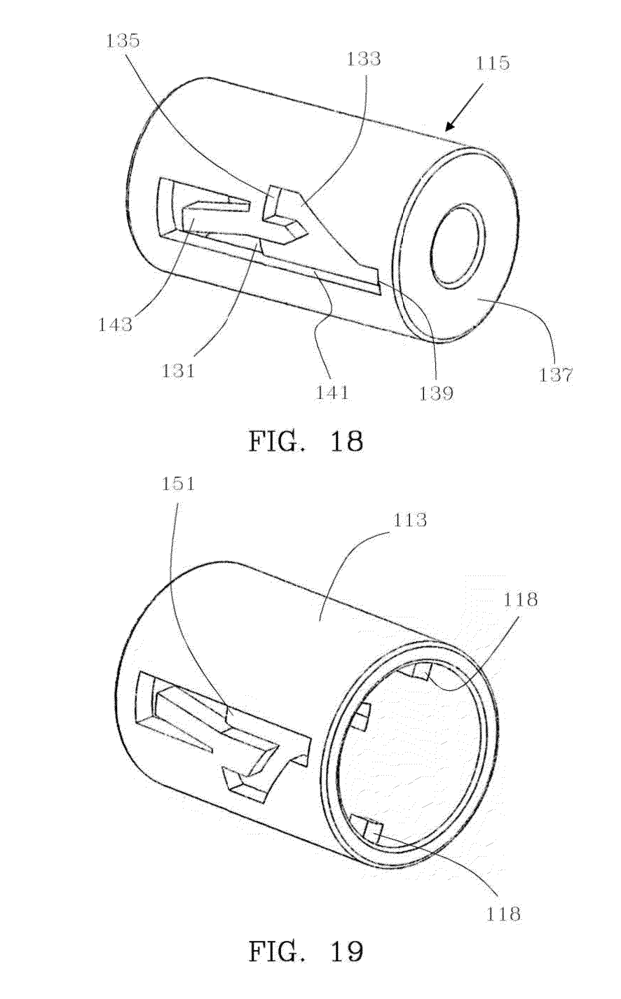

Referring to FIG. 18, the inner cylinder 115 of the telescoping region 116 can include at least one inner track 131 that can have any suitable shape, such as a J-shape. Referring to FIG. 19, the middle cylinder 113 can include at least one first protruding member 118 that can be integral with the inner surface of the middle cylinder 113, where the first protruding member 118 can be operable to translate within the at least one inner track 131 of the inner cylinder 115 from a pre-use position, to a retracted position, to an extended and locked position. The spring 119 (FIG. 17) can be disposed between the neck portion 114 and the inner cylinder 115 and can bias the telescoping region 116 in a generally distal direction. It will be appreciated that the at least one inner track 131 is shown by way of example only, where the at least one inner track 131 may have any suitable shape and configuration. It will be appreciated that one or a plurality of tracks may be incorporated to achieve a similar operation and it will be appreciated that the track and/or projection can be associated with any suitable component.

Referring to FIGS. 21-24, an example embodiment of the relative movement between the middle cylinder 113 and the inner cylinder 115 is shown. Referring to FIGS. 14 and 21, in a pre-use position, the first protruding member 118 of the middle cylinder 113 can be retained in a projection or lateral arm 133 of the at least one inner track 131, where the first protruding member 118 can abut a stop 135 and can be biased against the stop by the spring 119 (FIG. 14). Referring to FIGS. 16 and 22, during use, the distal surface 137 of the inner cylinder 115 can be retracted in response to contact with a patient's skin (not shown). The inner cylinder 115 can be retracted against the distal bias of the spring 119, where the telescoping region 116 can be retracted proximally and the needle 121 can be more fully exposed. With reference to FIG. 22, the first protruding member 118 can be advanced to the proximal end 139 of the at least one inner track 131, where the protruding member 118 can abut the proximal end 139 such that the telescoping region 116 cannot be further retracted. In an example embodiment, during use, the first protruding member 118 can travel from the stop 135 of the lateral arm 133 to the proximal end 139, where movement of the protruding member 118 can cause the middle cylinder 113 to rotate relative to the inner cylinder 115. When the first protruding member 118 is positioned at about the proximal end 139, the protruding member can be aligned with a substantially linear portion 141 of the at least one inner track 131. It will be appreciated that the example configurations, cylinders, and associated tracks can be used with a two cylinder design, a three cylinder design, or any suitable design.

Referring to FIG. 23, as a patient's skin is pulled away from the distal surface 137 of the inner cylinder 115, the spring 119 can be operable to extend the telescoping region 116 in a generally distal direction. As the inner cylinder 115 translates distally, the first protruding member 118 can travel within the substantially linear portion 141 of the at least one inner track 131 such that the first protruding member 118 comes into contact with a first living hinge 143 associated with the inner cylinder 115. The first living hinge 143 can biased radially into the pathway of the substantially linear portion 141, where the first protruding member 118 can flex the first living hinge 143 radially inward as shown in FIG. 23.

Referring to FIGS. 17 and 24, when the safety system 108 is removed from a patient's skin, the telescoping region 116 can be fully extended to a distal locked position such that the needle 121 is fully covered. Referring to FIG. 24, the inner cylinder 115 can be distally advanced by the spring 119 such that the first protruding member 118 can pass by the first living hinge 143 and the first living hinge 143 can return to a relaxed position, as illustrated. When the first living hinge 143 is in the relaxed position, as shown in FIG. 24, the first protruding member 118 can be permanently retained by a distal hinge surface 145 and a proximal track surface 147 such that the first protruding member 118 is unable to move in a proximal or distal direction. In this manner, the needle guard 108 can be configured for only single-use applications. In an example embodiment, the inner cylinder 115 can be substantially non-rotatable, where the middle cylinder 113, or other components, can be configured to rotate during operation, such that a patient will not feel any rotating components against their skin.

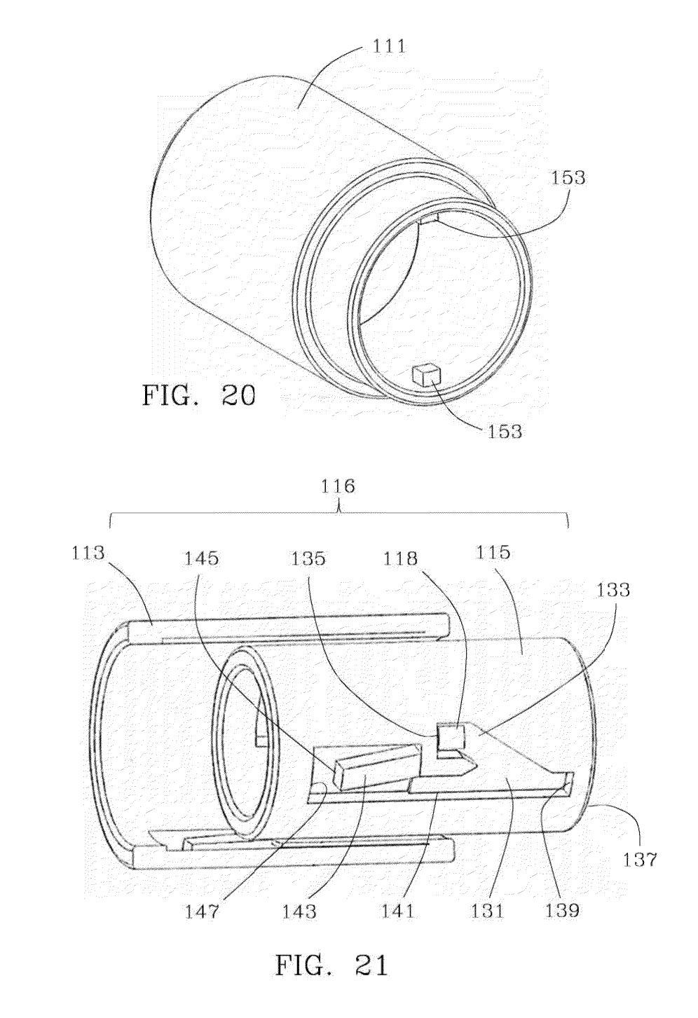

It will be appreciated that the telescoping region 116 can include any suitable number of telescoping sections. Each telescoping section can communicate as described with respect to the inner cylinder 115 and the middle cylinder 113. For example, referring to FIG. 19, the middle cylinder 113 can include at least one middle track 151 and, referring to FIG. 20, the outer cylinder 111 can include a second protruding member 153 that can be configured to engage the at least one middle track 151. In an example embodiment, the first protruding member 118 can travel within the at least one inner track 131 at the same time the second protruding member 153 can travel within the at least one middle track 151. In this manner, multiple telescoping components can be configured to move concomitantly. Any suitable number of telescoping components having any suitable relationship is contemplated. It will be appreciated that the telescoping region 116 can be configured such that the inner cylinder 115 does not rotate relative to a patient's skin, where such a configuration may improve patient comfort.

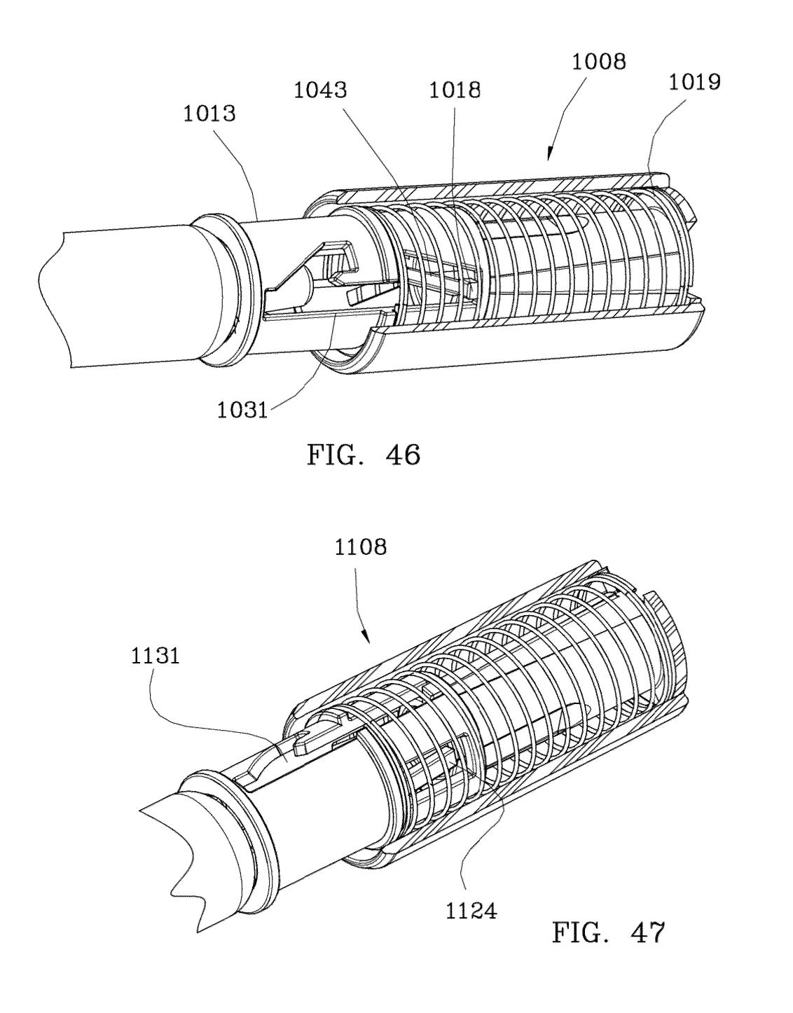

It will be appreciated that the embodiment illustrated in FIGS. 21-24 is shown by way of example only. For example, the embodiment of FIGS. 21-24 is illustrated with a spring 119 that biases the cylinders in a generally distal direction. In an alternate embodiment, as illustrated in FIG. 46, a non-reusable needle guard 1008 can include a spring 1019 that can be configured to bias one or a plurality of cylinders 1013 in a generally proximal direction. Such a configuration can be associated with movable and/or static cylinders or other such guard components. It will be appreciated that the track 1031, projection 1018, flexible hinge 1043, associated guidance or locking components can have a reverse, inverted, or mirror-image configuration to that illustrated in FIG. 21, for example. It may be beneficial to provide a compression spring or tension spring having any suitable configuration, where it will be appreciated that a track or associated mechanisms can be configured to accommodate such configurations.

The embodiment illustrated in FIG. 47 illustrates one embodiment of an alternate embodiment of a non-reusable needle guard 1108. It will be appreciated that the needle guard 1108 can function in substantially the same manner as needle guard 1008, however, the needle guard 1108 can include one or a plurality of lockout features 1024 that operate independently of a track 1131. It will be appreciated that the guidance and locking features can be integrated in to a single mechanism or can operate independently. It may be beneficial to separate or combine features depending upon design and use considerations. In one embodiment, the one or a plurality of locking features 1024 can operate in a manner consistent with the flexible hinges 424 shown in FIGS. 37 and 38.

Referring to FIG. 14, the safety system 108 can be coupled to the needle retaining device 128 in any suitable manner. In an example embodiment, the neck portion 114 can define an inner diameter 155 (FIG. 13), where the inner diameter 155 may be larger than the diameter of the head 157 of the needle retaining device 128. The outer cylinder 111 can be configured such that drawing the outer cylinder 111 proximally can tension the neck portion 114 such that the inner diameter 155 is reduced. Reducing the inner diameter 150 in this manner can securely or more securely couple the safety system 108 to the needle retaining device 128. In an alternate embodiment, the neck portion 114 can snap fit onto the needle retaining device 128, where the outer cylinder 111 can more securely couple the needle retaining device 128 with the safety system 108. Embodiments can be designed to fit in a standard 3 inch tub and 160 nest.

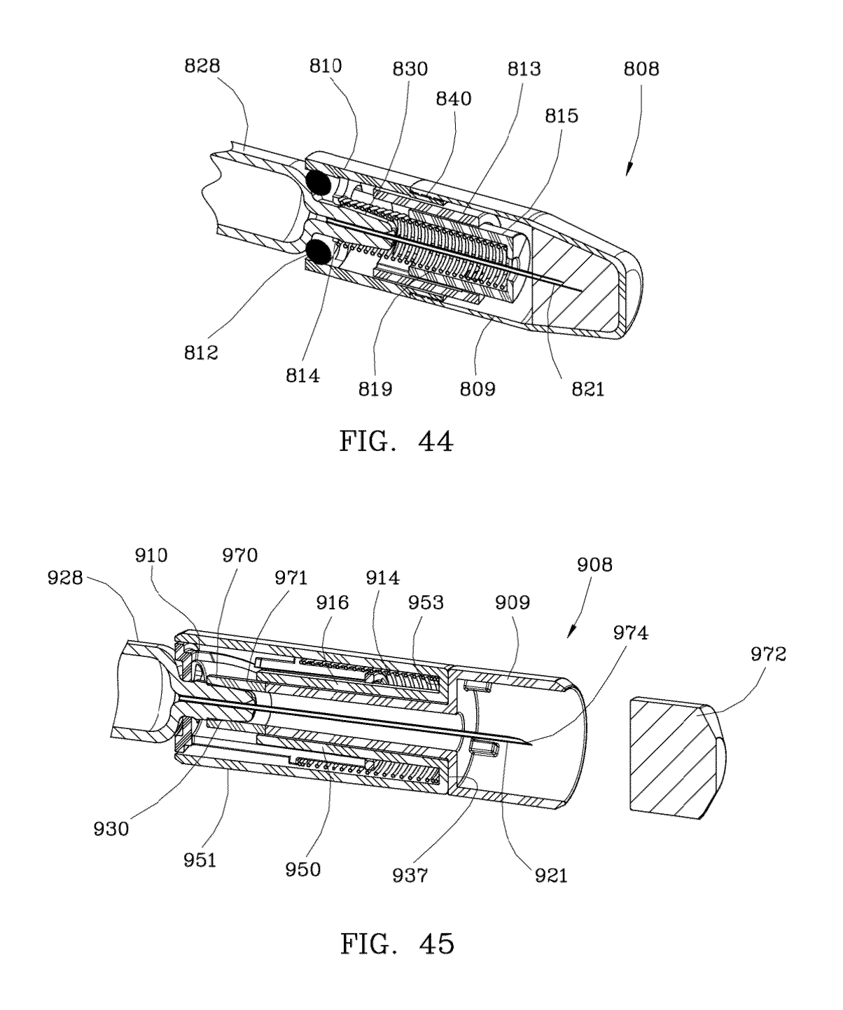

Referring to FIG. 44, an automatic non-reusable needle guard 808 can include a coupling cylinder 810 that can be attached to a needle retaining device 828, such as a standard syringe or pre-filled cartridge that can retain a needle 821. The coupling cylinder 810 can have a seal 812 and a neck portion 814. In an example embodiment, the neck portion 814 can be coupled with the neck 830 of the standard needle retaining device 828 with a snap fit, friction fit, or any other suitable coupling. It will be appreciated that needle guard 808 can have any suitable shape, size, or configuration. It will be appreciated that the coupling can be associated with a sterile barrier that can maintain the sterility of the needle 821 and a medicament or drug contained within a pre-filled syringe. The needle guard 808 can include a pre-use cap 809 during shipping or storage that can be removed just prior to use. The pre-use cap 809, as described in more detail herein, can be configured to create a sterile barrier by including a tortuous path or other suitable configuration, design, or construction.

The automatic non-reusable needle guard 808 can include a middle cylinder 813, and an inner cylinder 815, where the telescoping region can be biased distally with a spring 819. The needle guard 808 can be configured to transition from an initial retractable position, to a retracted position, to an extended and locked position. It will be appreciated that the needle guard 808 can include any suitable number of telescoping components, including telescoping cylinders, in accordance with systems described herein.

Still referring to FIG. 44, the needle 821 sterility of a multi-cylinder needle guard 808 design can be maintained by two seals, for example. The first sealing region can be between the proximal end of the automatic non-reusable needle guard 808 and the neck 830 of the standard needle retaining device 828. It will be appreciated that the seal 812 can include any suitable sealing element or component such as an O-ring, a co-molded seal, a seal mounted or glued on the needle guard 808, or the like. The second sealing region can be located at the junction between the exterior distal end of the coupling cylinder 810 and the inner proximal region of the pre-use cap 809. This second seal can include a tortuous path of protruding features 840 on the pre-use cap 809, the outer cylinder (not shown), or both. The tortuous path, which can be an effective sterility barrier, can be maintained by compressive forces resulting from the interference fit of the pre-use cap 809 and the coupling cylinder 810. In this manner, the needle guard 808 can be sealed at about the proximal end and at about the distal end such that an effective sterility barrier is provided. It will be appreciated that any suitable mechanism for providing a sterility barrier is contemplated, where any suitable seal, number of seals, seal configuration, or placement such as an additional elastomeric component, assembled or co-molded, is contemplated. The sterility barrier can include a barrier region comprising the interior of the needle guard 808, the first sealing region, the second sealing region, and/or any other suitable featured that can be configured to maintain the sterility of the needle, syringe, or medicament.

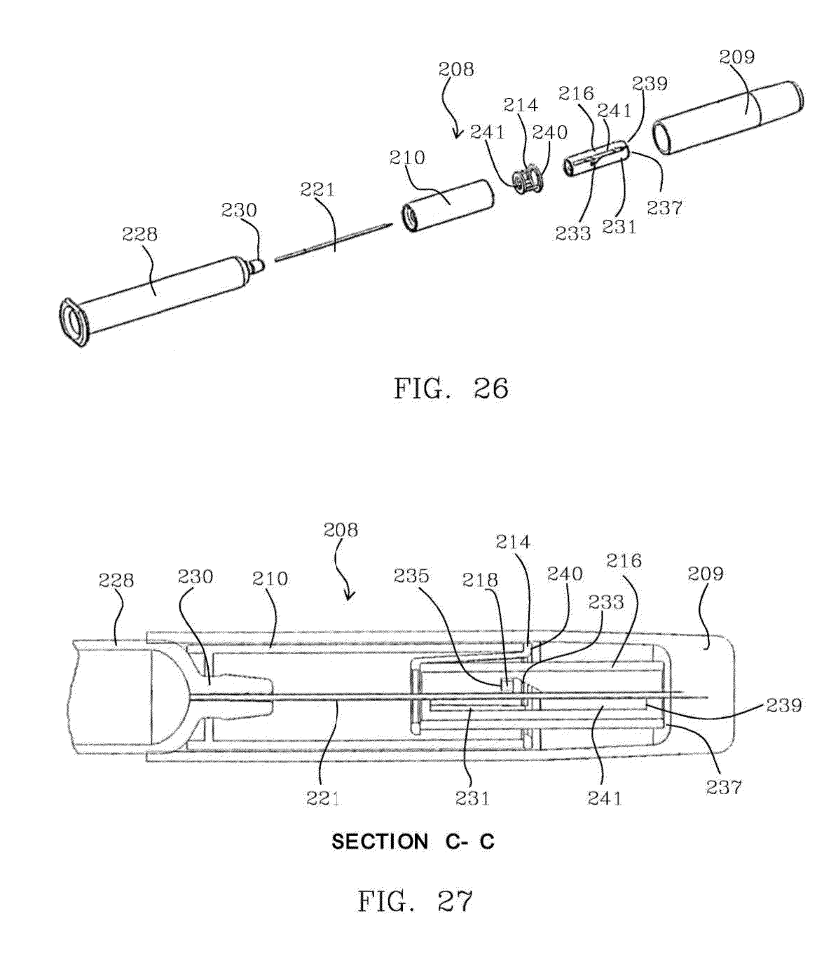

Referring to FIGS. 25-30, an automatic non-reusable needle guard 208 can include a coupling cylinder 210 (FIG. 26) that can be attached to a needle retaining device 228, such as a standard syringe or pre-filled cartridge that can retain a needle 221 (FIG. 26). The coupling cylinder 210 can be coupled with the neck 230 of the standard needle retaining device 228 with a snap fit, friction fit, or any other suitable coupling. It will be appreciated that needle guard 208 can have any suitable shape, size, or configuration. The needle guard can include a pre-use cap 209 (FIGS. 25-27) during shipping or storage that can be removed just prior to use. It will be appreciated that the coupling with the syringe can include a sterile barrier or boundary and the pre-use cap can be configured to maintain the sterility of the needle, syringe, or any other suitable device or component.

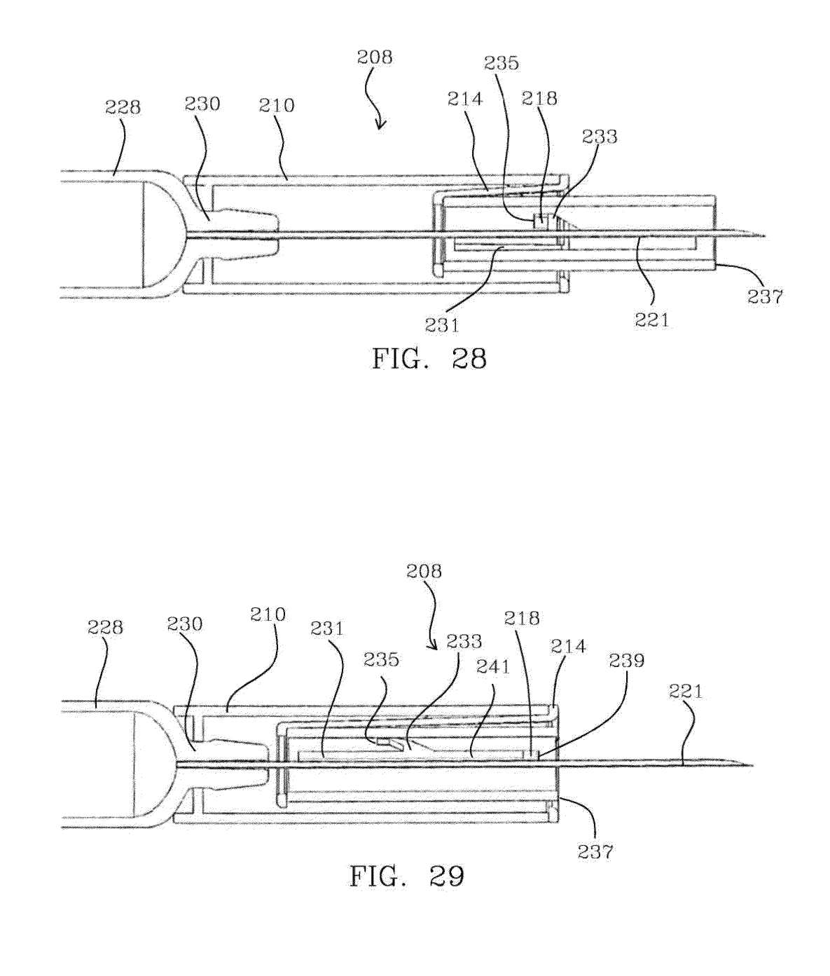

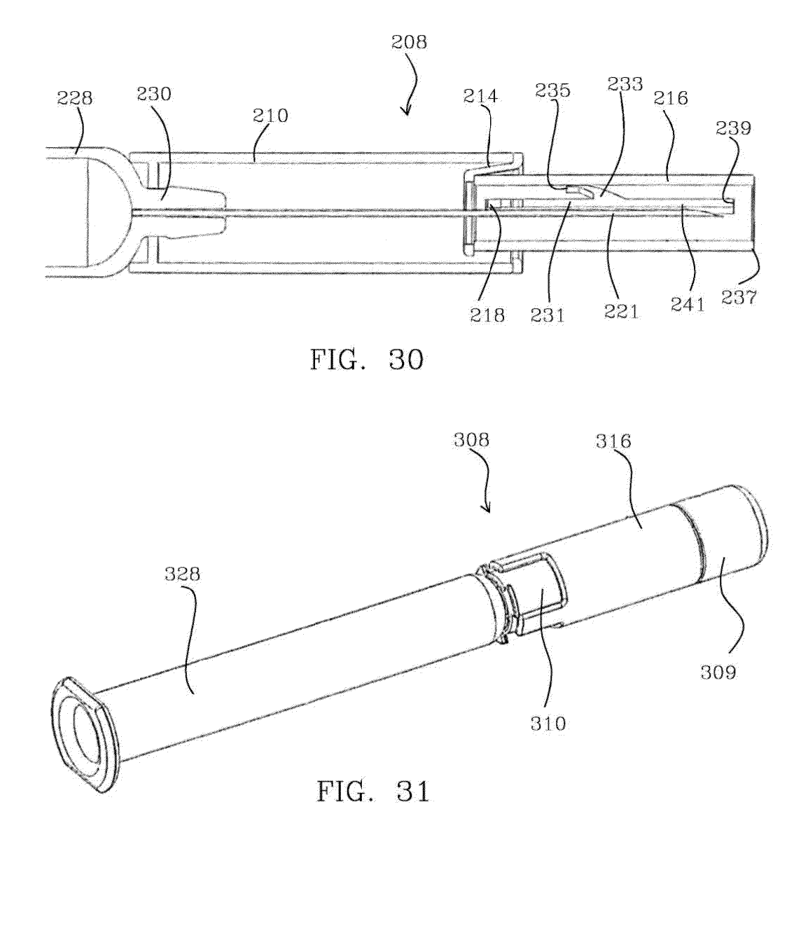

The automatic non-reusable needle guard 208 can include a movable guard 216, where the movable guard 216 can be biased distally with a tension spring 214. The movable guard 216 can be operable for movement from an initial retractable position (FIGS. 27 and 28), to a retracted position (FIG. 29), and to an extended position (FIG. 30). It will be appreciated that the movable guard 216 can include any suitable number of components, including telescoping cylinders, in accordance with systems described herein. The tension spring 214 can be substantially or partially constructed from an elastic material.

Referring to FIG. 26, the movable guard 216 can include at least one inner track 231 that can have any suitable shape, such as a J-shape or a linear track having a radial projection. Referring to FIG. 27, the coupling cylinder 210 can include at least one protruding member 218 that can be integral with the inner surface of the coupling cylinder 210, where the first protruding member 218 can be operable to translate within the at least one inner track 231 from a pre-use position, to a retracted position, to an extended position. The tension spring 214 can include a distal end 240 that can engage the distal end of the coupling cylinder 210 and a proximal end 241 that can support the proximal end of the movable guard 216. The tension spring 214 can bias the movable guard 216 in a generally distal direction. It will be appreciated that the at least one inner track 231 is shown by way of example only, where the at least one inner track 231 can have any suitable shape or configuration. It will be appreciated that one or a plurality of tracks may be incorporated to achieve a similar operation and it will be appreciated that the track and/or projection can be associated with any suitable component. It will be appreciated that the tension spring is shown by way of example only, where the tension spring can have any suitable orientation or configuration.

Referring to FIGS. 27-30, an example embodiment of the relative movement between the movable guard 216 and the coupling cylinder 210 is shown. Referring to FIGS. 27 and 28, in a pre-use position, the first protruding member 218 of the coupling cylinder 110 can be retained in a lateral arm 233 of the at least one inner track 231, where the first protruding member 218 can abut a stop 235 and can be biased against the stop by the tension spring 214. Referring to FIG. 29, during use, the distal surface 237 of movable guard 216 can be retracted proximally in response to contact with a patient's skin (not shown). The movable guard 216 can be retracted against the distal bias of the tension spring 214 such that the movable guard 216 is retracted proximally and the needle 221 is more fully exposed. With reference to FIG. 29, the first protruding member 218 can be advanced to the proximal end 239 of the at least one inner track 231, where the protruding member 218 can abut the proximal end 239 such that the movable guard 216 cannot be further retracted. In an example embodiment, during use, the first protruding member 218 can travel from the stop 235 of the lateral arm 233 to the proximal end 239, where movement of the protruding member 218 can cause the movable guard 216 to rotate relative to the coupling cylinder 210. When the first protruding member 218 is positioned at about the proximal end 239, the protruding member 218 can be aligned with a substantially linear portion 241 of the at least one inner track 231. It will be appreciated that embodiments are contemplated that can incorporate an outer track or no track.

Referring to FIG. 30, when the safety system 208 is removed from a patient's skin, the movable guard 216 can be fully extended to a distal covered position such that the needle 221 is fully or substantially covered. Referring to FIG. 30, the movable guard 216 can be distally advanced by the tension spring 214 such that the tension spring 214 is in a substantially relaxed position in the distal-most position. It will be appreciated that the safety system 208 can lock the guard in the distal position in accordance with embodiments described herein.

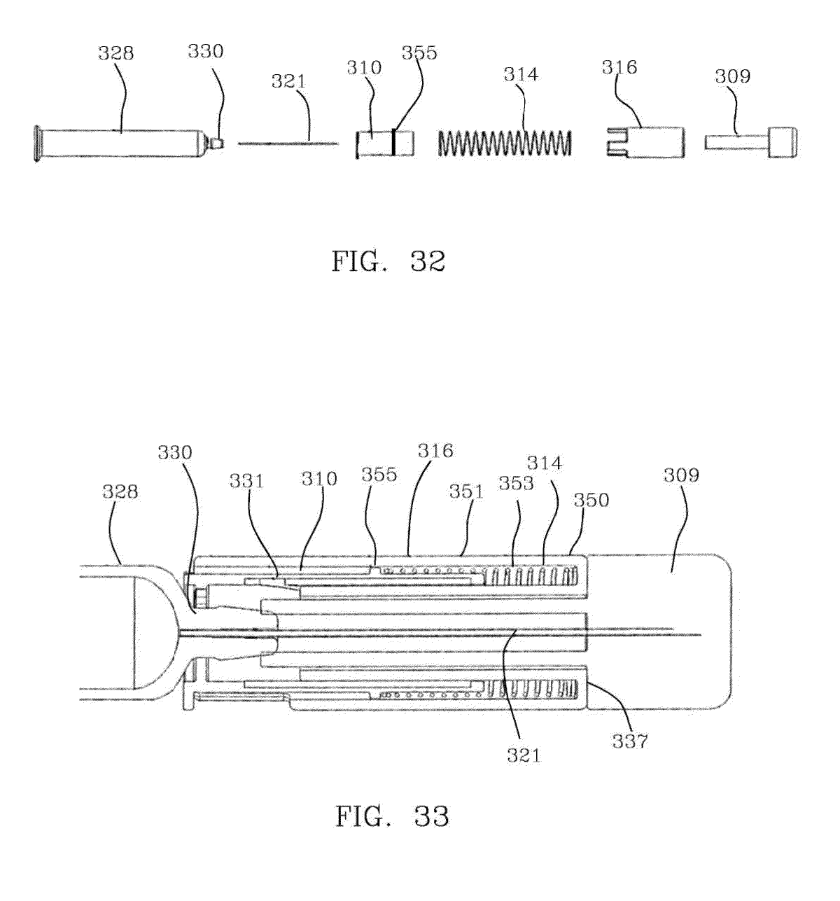

Referring to FIGS. 31-36, an automatic non-reusable needle guard 308 can include a coupling cylinder 310 that can be attached to a needle retaining device 328, such as a standard syringe or pre-filled cartridge that can retain a needle 321. The coupling cylinder 310 can be coupled with the neck 330 of the standard needle retaining device 328 with a snap fit, friction fit, or any other suitable coupling. It will be appreciated that needle guard 308 can have any suitable shape, size, or configuration. The needle guard can include a pre-use cap 309 during shipping or storage that can be removed just prior to use.

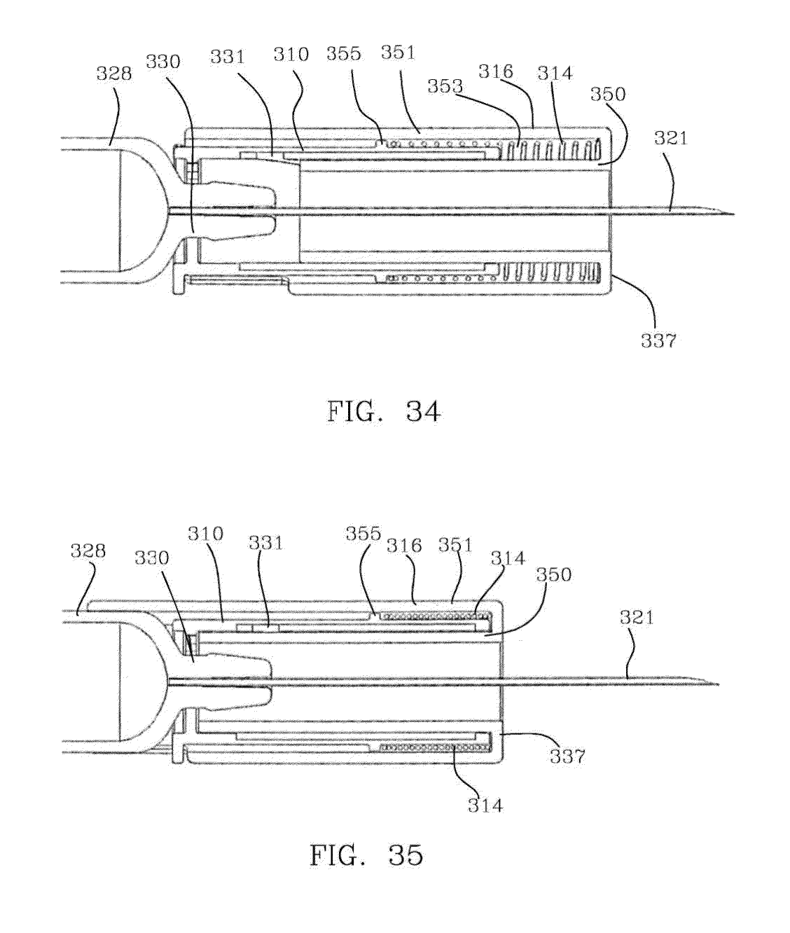

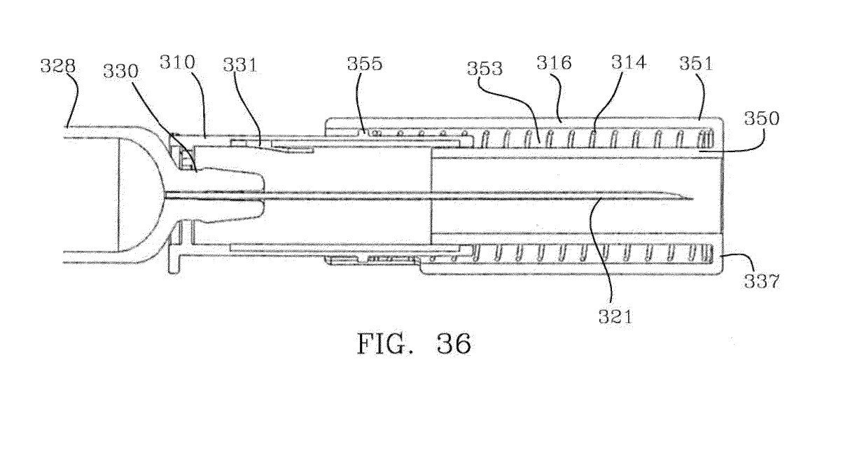

The automatic non-reusable needle guard 308 can include a movable guard 316, where the movable guard 316 can be biased distally with a spring 314. The movable guard 316 can be operable for movement from an initial retractable position (FIGS. 33 and 34), to a retracted position (FIG. 35), and to an extended position (FIG. 36). It will be appreciated that the movable guard 316 can include any suitable number of components, including telescoping cylinders, inner cylinders, outer cylinders, or any other component in accordance with systems described herein. The movable guard 316 can include a first annular portion 350 that can be positioned within the coupling cylinder 310 and a second annular portion 351 that can be positioned outside the coupling cylinder 310. The first annular portion 350, the second annular portion 351, and a distal end 337 of the movable guard 316 can define a cavity 353. The coupling cylinder 310 can include an annular band 355, where the spring 314 can be positioned within the cavity 353 between the annular band 355 and the distal end 337. The spring 314 can be substantially concealed by the second annular portion 351 of the movable guard 316 during use, which may reduce the risk of interference between the spring 314 and the needle 321.

Referring to FIG. 33-36, the coupling cylinder 310 can include at least one inner track 331 that can have any suitable shape, such as a J-shape or a linear track having a radial projection. Referring to FIG. 34, the coupling cylinder 310 can include a protruding member (not shown) that can be integral with the movable guard 316, where the protruding member can be operable to translate within the at least one inner track 331 from a pre-use position, to a retracted position, and to an extended position in a similar manner as described with respect to alternate embodiments. The spring 314 can bias the movable guard 316 in a generally distal direction. It will be appreciated that the at least one inner track 331 is shown by way of example only, where the at least one inner track 331 may have any suitable shape or configuration. It will be appreciated that one or a plurality of tracks may be incorporated to achieve a similar operation and it will be appreciated that the track and/or projection can be associated with any suitable component. It will be appreciated that the compression spring is shown by way of example only, where the compression spring may have any suitable orientation or configuration.

Referring to FIGS. 33-36, an example embodiment of the relative movement between the movable guard 316 and the coupling cylinder 310 is shown. Referring to FIGS. 33 and 34, in a pre-use position, the protruding member (not shown) of the movable cylinder 316 can be retained in the at least one inner track 331. Referring to FIG. 35, during use, the distal end 337 of movable guard 316 can be retracted in response to contact with a patient's skin (not shown). The movable guard 316 can be retracted against the distal bias of the tension spring 314 such that the movable guard 316 is retracted proximally and the needle 321 is more fully exposed.

Referring to FIG. 36, when the safety system 308 is removed from a patient's skin, the movable guard 316 can be fully extended to a distal covered position such that the needle 321 is fully covered. Referring to FIG. 36, the movable guard 316 can be distally advanced by the spring 314. It will be appreciated that the safety system 308 can lock the movable guard 316 in the distal position in accordance with versions described herein. It will be appreciated that any suitable configuration having a fixed or stationary inner cylinder and a movable outer cylinder is contemplated.

Referring to FIG. 45, an automatic non-reusable needle guard 908 can include a coupling cylinder 910 that can be attached to a needle retaining device 928, such as a standard syringe or pre-filled cartridge that can retain a needle 921. The coupling cylinder 910 can be coupled with the neck 930 of the standard needle retaining device 928 with a snap fit, friction fit, or any other suitable coupling. It will be appreciated that needle guard 908 can have any suitable shape, size, or configuration. The needle guard can include a pre-use cap 909 during shipping or storage that can be removed just prior to use.

The automatic non-reusable needle guard 908 can include a movable guard 916, where the movable guard 916 can be biased distally with a spring 914. The movable guard 916 can be operable for movement from an initial retractable position, to a retracted position, to an extended position. It will be appreciated that the movable guard 916 can include any suitable number of components, including telescoping cylinders, inner cylinders, outer cylinders, or any other component in accordance with systems described herein. The movable guard 916 can include a first annular portion 950 that can be positioned within the coupling cylinder 910 and a second annular portion 951 that can be positioned outside the coupling cylinder 910. The first annular portion 950, the second annular portion 951, and a distal end 937 of the movable guard 916 can define a cavity 953. The spring 914 can be substantially concealed by the second annular portion 951 of the movable guard 916 during use.

The needle guard 908 can include, for example, two sterile seals that can be positioned at about the distal and proximal ends of the automatic non-reusable needle guard 908 such that the sterility of the needle 921 can be maintained. One embodiment of a proximal seal 970 includes an elastomeric portion 971 at about the proximal end of the pre-use cap 909. The proximal seal 970 can be press-fit, co-molded, insert molded, or otherwise mechanically attached or bonded to the pre-use cap 909. The outside diameter of the elastomeric proximal seal 970 of the pre-use cap 909 can be sized such that it can be applied to the neck 930 of the standard needle retaining device 928 within the coupling cylinder 910 of the automatic non-reusable needle guard 908. The inside diameter of the elastomeric proximal seal 970 of the pre-use cap 909 can be sized to fit over the neck 930 of the standard needle retaining device 928, where the compression of the elastomeric region 971 of the pre-use cap 909 on the neck 930 can maintain a sterile seal. The proximal seal 970 can be used in conjunction with a distal seal 972 that can maintain the fluid in the standard needle retaining device 928.

A distal seal 972 can plug the distal end of the pre-use cap 909. When the pre-use cap 909 is seated fully on the neck 930 of a standard needle retaining device 928, the needle 921 can penetrate into the distal seal 928 such that the fluid in the attached syringe or cartridge is retained therein. When used in conjunction with the sealed proximal couplings described herein, for example, the distal seal 972 can maintain the sterility of the needle 921. The distal seal 972 can be configured from silicone, TPE, or other elastomeric component, where the distal seal 972 can be bonded to the pre-use cap 909 by co-molding or insert molding, for example. In such an embodiment, the sterile barrier can be maintained by the bond between the distal seal 972 and the pre-use cap 909.

The distal seal 972 can be formed in a separate molding operation from the pre-use cap 909 and can be placed into the pre-use cap 909 as a separate step in the assembly of the needle guard 908 to the standard needle retaining device 928. This can allow the needle tip 974 to be inspected for potential damage or contamination after the assembly of the automatic non-reusable needle guard 908 to the standard needle retaining device 928. After the assembly of the distal seal 972 to the pre-use cap 909, the sterile barrier at the distal end of the pre-use cap 909 can be maintained by the compressive forces between the distal seal 972 and the inner surface of the pre-use cap 909. It will be appreciated that various raised surface features (not shown) on the distal seal 972 or the inner surface of the pre use cap 909 can facilitate the maintenance of a sterile barrier by locally increasing the pressure between the distal seal 972 and the inner distal surface of the non-reusable needle guard 908. The non-reusable needle guard 908 can also include undercuts (not shown) at the distal end that can provide a mechanical mechanism for preventing the distal movement of the distal seal 972.

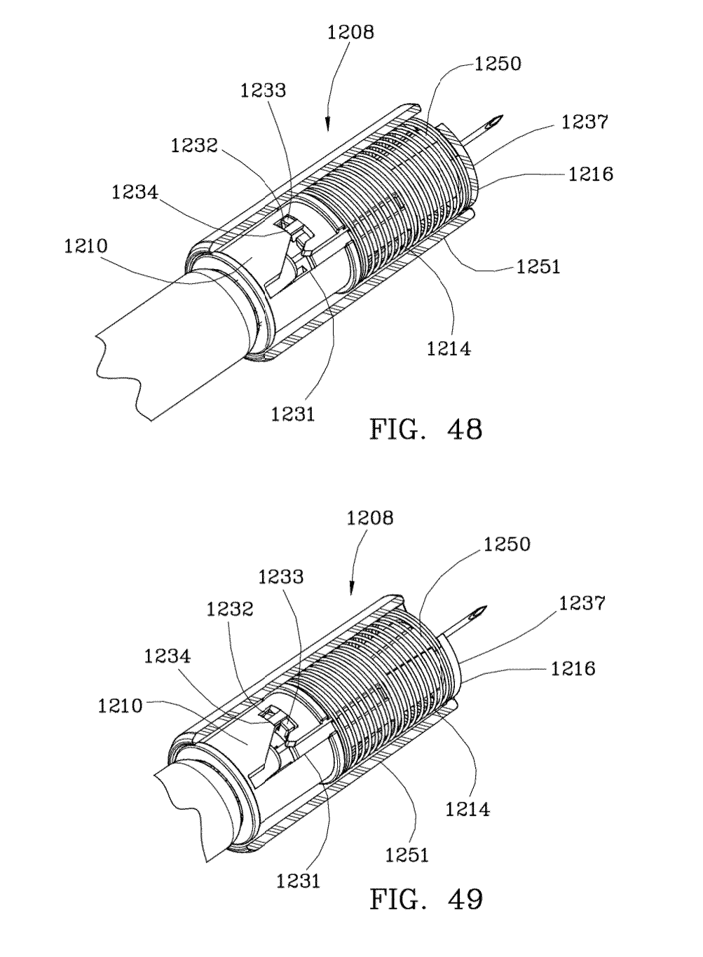

Referring to FIGS. 48-50, an automatic non-reusable needle guard 1208 can include a movable guard 1216, where the movable guard 1216 can be biased distally with a spring 1214. The movable guard 1216 can be operable for movement from a first initial locked position (FIG. 48), to an operative second position (FIG. 49) as will be described in more detail herein. It will be appreciated that the needle guard 1208, movable guard 1216, or the like can include any suitable components, elements or mechanisms that can be configured to prevent premature activation or deployment of the needle guard 1208. It may be beneficial to provide a mechanical structure or design configuration that can prevent the accidental deployment of the needle guard 1208 prior to use.

The movable guard 1216 can include a first annular portion 1250 that can be positioned within the coupling cylinder 1210 and a second annular portion 1251 that can be positioned outside the coupling cylinder 1210. The first annular portion 1250, the second annular portion 1251, and a distal end 1237 of the movable guard 1216 can define a cavity. The spring 1214 can be substantially concealed by the second annular portion 1251 of the movable guard 1216, and retained within the cavity, during use. The outer or second annular portion 1210 can be designed such that it can slide over the needle retaining device 1228 during deployment for enhanced guidance.

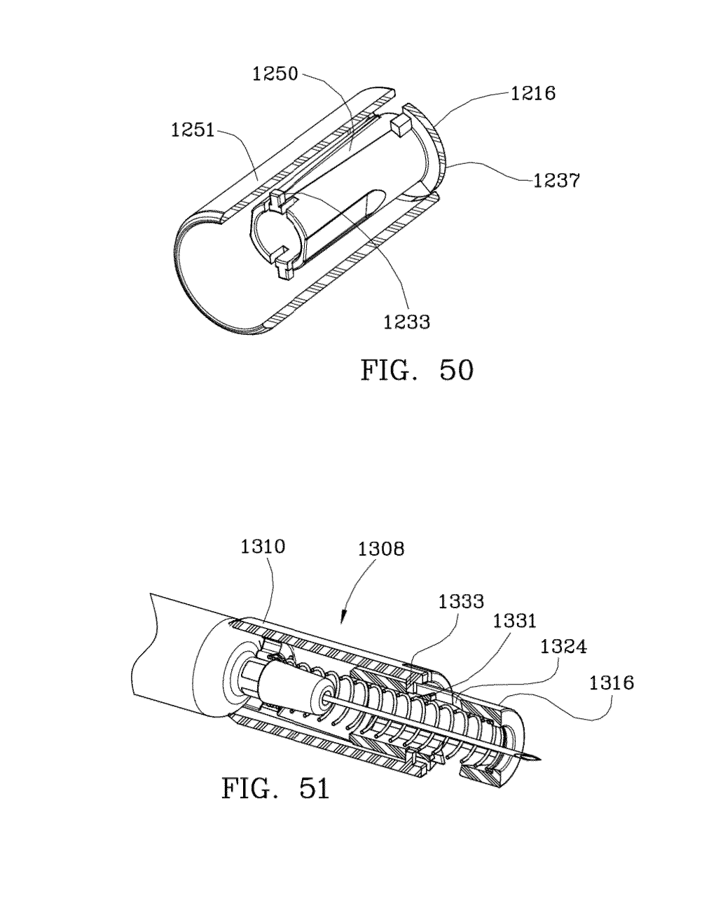

Referring to FIGS. 48 and 49, the coupling cylinder 1210 can include at least one inner track 1231 that can have any suitable shape, such as a J-shaped track having a lateral arm 1232. Referring to FIG. 50, the movable guard 1216 can include at least one protruding member 1233 that can be integral with the movable guard 1216, where the protruding member 1233 can be operable to translate within the at least one inner track 1231 in a similar manner as described with respect to alternate embodiments. The lateral arm 1232 of the at least one inner track 1231 can include a ramp 1234 that, in one embodiment, must be overcome with rotational force for the needle guard 1208 to become operable. The protruding member 1233 can be flexible, a living hinge, or otherwise configured to pass over the ramp 1234 when sufficient force is applied. In this manner, the needle guard 1208 can be prevented from prematurely activating prior to use. The spring 1214 can bias the movable guard 1216 in a generally distal direction. It will be appreciated that the at least one inner track 1231 is shown by way of example only, where the at least one inner track 1231 or lateral arm 1232 may have any suitable shape or configuration. It will be appreciated that one or a plurality of tracks can be incorporated to achieve a similar operation and it will be appreciated that the track and/or projection can be associated with any suitable component. It will be appreciated that the spring is shown by way of example only, where the spring may have any suitable orientation or configuration. It will be appreciated that the ramp and/or protection can be flexible where, for example, a ramp can be laterally moved by a substantially rigid projection during operation.

Still referring to FIGS. 48-50, the non-reusable needle guard 1208 can incorporate numerous features to prevent the accidental deployment of the movable guard 1208 to the extended irreversible position. Features of the at least one inner track or J-track can be modified such that a loading position can exist where the movable guard 1216 can be prevented from the proximal movement necessary to overcome this initial state. The raised ramp 1234 feature can be molded into the coupling cylinder 1210 such that the first protruding member 1233 of the movable guard 1216 can be prevented from moving to the second position (FIG. 49) until the first protruding member 1233 on movable guard 1216 is rotated across the ramp 1234 into the second position. Alternatively, the raised ramp feature can be located on the movable guard (not shown), with the first protruding member located on the coupling cylinder (not shown). The raised ramp feature can require a torque threshold to overcome the ramp, where the rotation of the first protruding member across the ramp can require flexion in the movable guard, the coupling cylinder, or both, for the first protruding member to cross the ramp. The ramp can be designed or configured such that the application of torque can allow the first protruding member to cross the ramp in only one direction, with an interference height abutting the first protruding member, that can prevent the return of the first protruding member to the first position. Such a premature deployment feature can be used in a manufacturing environment only, where the loading state can facilitate attachment of the automatic non-reusable needle guard to a standard needle retaining device, but where the device can be stored with the premature deployment feature defeated or otherwise inactive. Alternatively, the premature deployment feature can be left engaged just prior to use, where the needle guard is in the first position, where the user can be required to rotate the movable guard or otherwise transition the needle guard to the second position prior to the use of the injection device.

Referring to FIGS. 51-54, it will be appreciated that any suitable components or mechanisms can be provided in a needle guard to prevent premature deployment. Referring to FIG. 51, one embodiment of an automatic non-usable needle guard 1308 can include a movable guard 1316 that can have a longitudinally oriented flexible ramp 1324 positioned within a channel or track 1331. A coupling cylinder 1310 can include a projection 1333 that can engage the track 1331, where prior to use of the needle guard 1308 the projection 1333 can be required to advance past the flexible ramp 1324. In this manner, the ramp 1324 can prevent premature deployment by retaining the projection 1333 until sufficient force is applied. It will be appreciated that any suitable ramp feature can creates a loading position in-line, or otherwise, with the track or channel features on the coupling cylinder. A raised ramp, with a force threshold consistent with embodiments described herein, can prevent the accidental deployment of the automatic non-reusable needle guard into the extended irreversible position, provided that accidental forces applied to the movable guard are below the ramp threshold. It will be appreciated that a plurality of safety mechanism or structures are contemplated if desirable. As described herein, the safety mechanism can be engaged only during a manufacturing or shipping environment, or can be engaged just prior to use by a user.

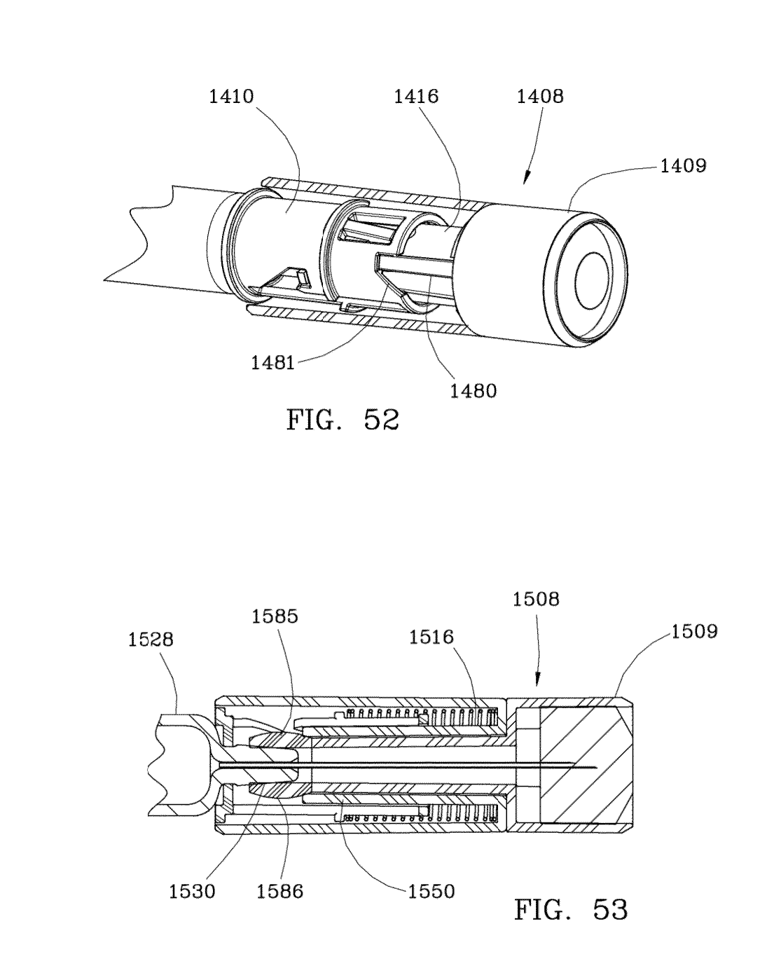

Referring to FIG. 52, an alternate mechanism for preventing premature deployment is illustrated. An non-reusable needle guard 1408 can include a coupling cylinder 1410, a movable guard 1416, and an end cap 1409, where the end cap 1409 can be configured to prevent relative movement between the coupling cylinder 1410 and the movable guard 1416 until just prior to use. The end cap 1409 can include a projection or arm 1480 that can abut or engage a stop 1481 that can be defined by the coupling cylinder 1410. The arm 1480 can physically prevent relative movement between the coupling cylinder 1410 and the movable guard 1416, where upon removal of the end cap 1409 the needle guard 1408 can be operated in accordance with embodiments described herein. In example embodiments, the interference of the arm 1480 and the coupling cylinder can also prevent rotational movement of the movable guard 1416. With the proximally extending finger or arm 1480 preventing relative rotation between the coupling cylinder 1410 and the movable guard 1416, the movable guard 1416 cannot follow the track 1431 and therefore cannot allow the movable guard 1416 to become displaced from the first pre-use position. An additional sloped wall on the stop 1481 can facilitate the removal of the pre-use cap 1409 by urging the pre-use cap distally as the end cap 1409 is rotated in the direction of the sloped stop 1481. Alternatively, the sloped wall can be omitted but the stop can include an extended abutting surface (not shown) on the coupling cylinder where the end cap can still be rotated to facilitate removal, but without the potential advantage of the sloped wall.

Referring to FIG. 53, an alternate mechanism for preventing premature deployment is illustrated. A non-reusable needle guard 1508 can include a pre-use cap 1509 that can include a proximal end section 1585 that can be made from an elastomeric material, where the proximal end section 1585 can include a friction bump 1586 located at about the proximal end of the end cap 1509. This friction bump 1586 can be designed such that it is small enough to pass through the first annular portion 1550 of a movable guard 1516 without interference. In an alternate embodiment, there can be interference between the first annular portion 1550 and the movable guard 1516, where such interface can be overcome by compressing the bump 1586. The change in geometry of the proximal end section 1585 of the pre-use cap 1509 can occur when the proximal end portion 1585 of the end cap 1509 stretches over the neck 1530 of a standard needle retaining device 1528. The proximal end section can prevent the proximal motion of the movable guard 1516 because of the interference between the bump 1586 of proximal end section 1585 and the first annular portion 1550. Force applied on the pre-use cap 1509 in the direction of the neck 1530 can further compress the elastomeric portion of the pre-use cap 1509, which can cause further interference and resistance to motion. Once the pre-use cap 1509 is removed and the friction bump 1586 is no longer present, the lockout mechanism can function as described in accordance with embodiments described herein.

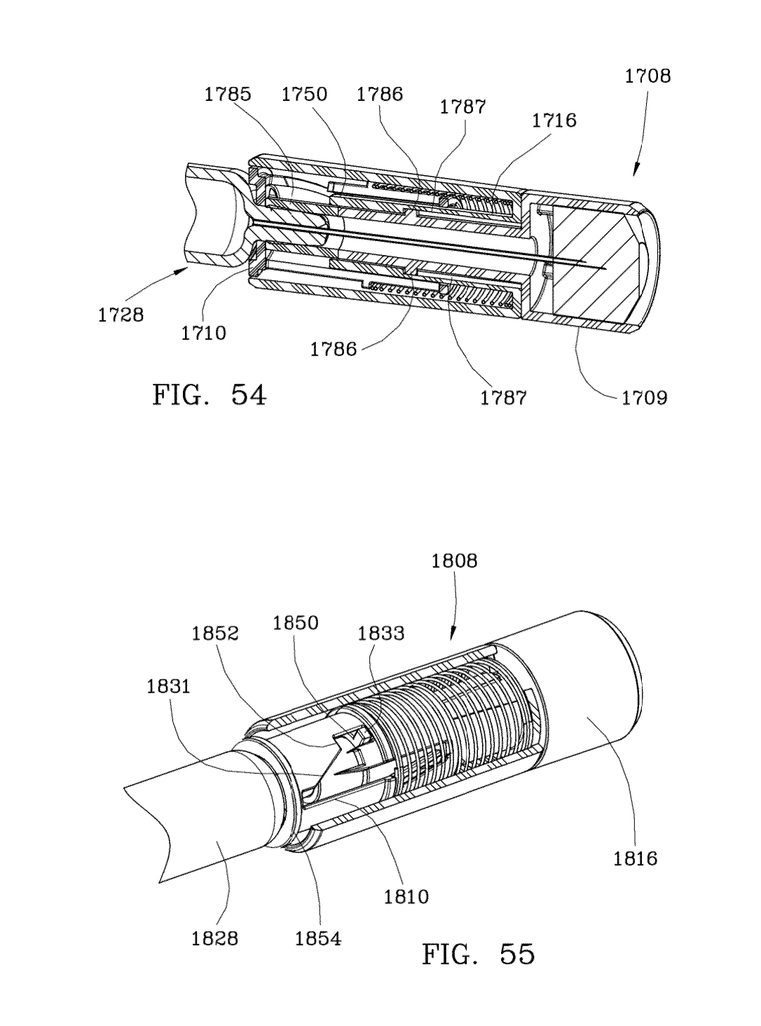

Referring to FIG. 54, an alternate mechanism that can prevent premature deployment is illustrated. An automatic non-reusable needle guard 1708 is shown that can include a pre-use cap 1709 that can incorporate one or more protruding members 1786. The protruding members 1786 can engage one or more corresponding internal pathways 1787 on the first annular portion 1750 of a movable guard 1716 such that longitudinal motion of the movable guard 1716 can be impeded until the cap 1709 is removed. Motion of the movable guard 1716 can be prevented by the countervailing rotational biases of the pathways 1787, which can prevent disengagement from the initial pre-use position due to the resistance of the protruding members 1786. The pathways 1787 can have a substantially J-shaped configuration, or any other suitable configuration, such that the cap 1709 can be required to be rotated or moved in a pre-determined motion relative to the movable guard 1716 before the cap 1709 can be released. Once the cap 1709 is released, the needle guard 1708 can operate in accordance with embodiments described herein.

The cap 1709 can also include a proximal end 1785 that can extend such that the proximal end can abut a coupling cylinder 1710 of the needle guard. The abutment between the proximal end 1785 and the coupling cylinder 1710 can prevent proximal force applied to the cap 1709 from deploying the needle guard 1708. The cap 1709 can have any suitable length or dimensions such that the cap 1709 can be required to be removed prior to the needle guard 1708 being used. The proximal end 1785 of the pre-use cap can include rigid or elastomeric elements that can be integrated or separate from any retaining features at the proximal end 1785 of the pre-use cap 1709. It will be appreciated that such features can be provided on the movable guard 1716, the coupling cylinder 1710, or any suitable component.

Still referring to FIG. 54, the automatic non-reusable needle guard 1708 can incorporate numerous features to facilitate attachment of the needle guard 1708 to the neck 1730 of a standard needle retaining device 1728, while at the same time preventing movement of the mechanism from the first pre-use position. As illustrated, the proximal end 1785 of the pre-use cap 1709 can abut the coupling cylinder 1710 in the first pre-use configuration. During assembly, the user can push or otherwise urge the needle guard 1708 onto the standard needle retaining device 1728 by pushing on the end cap 1709. The length of the end cap 1709 can be such that applying force to the end cap 1709 can engage the coupling cylinder 1710 with the neck 1730 without triggering the movable guard 1716. This configuration, alone or in combination with any suitable feature that can be used to prevent accidental deployment of the non-reusable needle guard 1708, can allow the needle guard 1708 to be affixed to the neck 730 of a standard needle retaining device 1728.

Referring to FIG. 55, an alternate mechanism that can prevent premature deployment during assembly is illustrated. An automatic non-reusable needle guard 1808 is shown that can include a movable guard 1816 having a projection 1833. A coupling cylinder 1810 can include a track 1831, where prior to use of the needle guard 1808 the projection 1833 can be retained in a lateral arm 1850 of the track 1831. It will be appreciated that the track 1831 can function in the manner described herein, where the lateral arm 1850 can prevent premature deployment of the needle guard 1808 during assembly. In one embodiment, during assembly, the movable guard 1816 can be proximally advanced such that the projection 1833 is urged against a stop 1852 of the track 1831. The stop 1852 can be used to transfer mechanical energy to the coupling cylinder 1810 such that the coupling cylinder 1810 can be engaged with a neck (not shown) of a needle retaining device 1828. Once the needle guard 1808 is attached, the projection 1833 can transition into the track 1831 such that the needle guard can function in accordance with embodiments described herein. For example, the projection can be transitioned from the first indented portion of the track 1831, as illustrated in FIG. 54, to the second indented portion of the track 1831. It will be appreciated that any suitable feature or configuration associated with a track or channel of a needle guard can be designed to prevent premature deployment during assembly. The needle guard 1808 can also include an annular projection or ridge 1854 at the proximal end of the coupling cylinder 1810 that can be gripped by an assembly fixture (not shown) when the automatic non-reusable needle guard 1808 is moved proximally to attach to the neck (not shown) of the standard needle retaining device 1828. Use of the ridge 1854 can prevent, in one embodiment, the application of any forces to a pre-use cap or movable guard, which can help prevent the accidental activation of the needle guard during assembly. After assembly of the needle guard to the standard needle retaining device 1828, the position of the movable guard 1816 inside the track 1831 on the coupling cylinder 1810 can be adjusted to move the needle guard 1808 into an initial usable state or second use position.

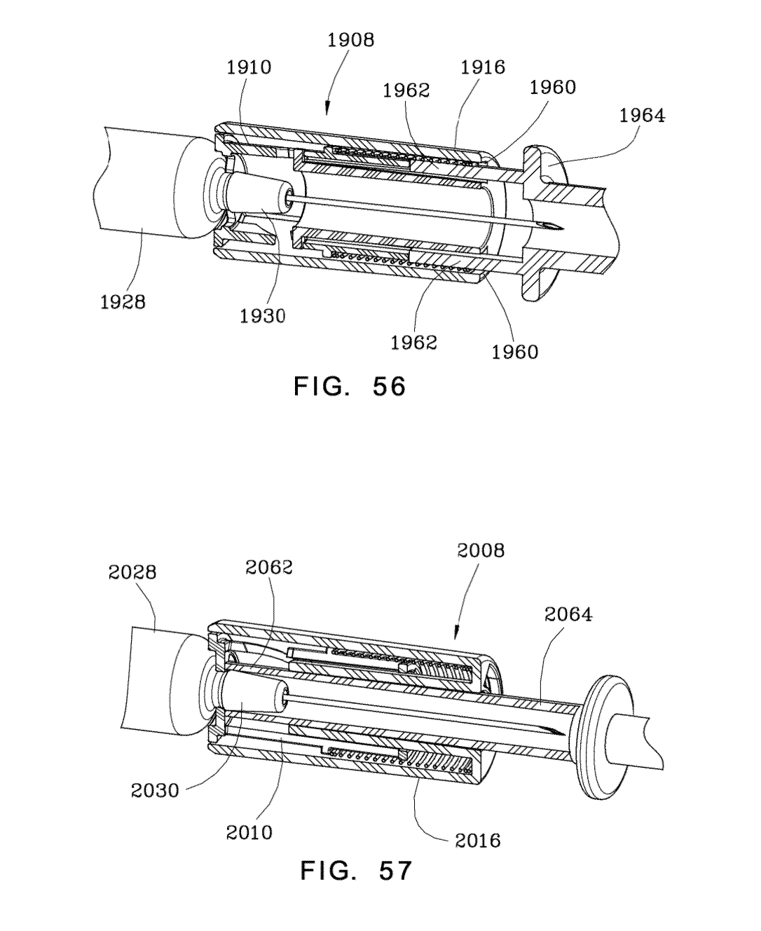

Referring to FIG. 56, an alternate mechanism that can prevent premature deployment during assembly is illustrated. An automatic non-reusable needle guard 1908 is shown that can include a movable guard 1916 associated with a coupling cylinder 1910. The needle guard 1908 can include one or a plurality of apertures 1960 that can be defined by the movable guard 1916 and can be configured to accept the arms 1962 of an assembly tool 1964. During assembly, the arms 1962 of the assembly tool 1964 can pass through the one or a plurality of apertures 1960 and can engage the coupling cylinder 1910. Force applied to the coupling cylinder 1910 can engage the needle guard 1908 with a neck 1930 of a needle retaining device 1928 to complete the assembly. The assembly tool 1964 can be configured to apply sufficient linear force necessary to attach the needle guard 1908 to the standard needle retaining device 1928. Application of the assembly force directly to the coupling cylinder 1910, rather than to the movable guard 1916, can prevent the accidental activation of the needle guard during assembly. It will be appreciated that an assembly tool can have any suitable configuration and such that the needle guard can be assembled without deploying the needle guard mechanism.

Referring to FIG. 57, an alternate mechanism that can prevent premature deployment during assembly is illustrated. An automatic non-reusable needle guard 2008 is shown that can include a movable guard 2016 associated with a coupling cylinder 2010. The needle guard 2008 can be configured to accept an assembly tool 2064. During assembly, the proximal end 2062 of the assembly tool 2064 can pass through the needle guard 2008 and can engage the coupling cylinder 2010. Force applied to the coupling cylinder 2010 can engage the needle guard 2008 with a neck 2030 of a needle retaining device 2028 to complete the assembly. The assembly tool 2064 can be configured to apply sufficient linear force necessary to attach the needle guard 2008 to the standard needle retaining device 2028. Application of the assembly force directly to the coupling cylinder 2010, rather than to the movable guard 2016, can prevent the accidental activation of the needle guard 2008 during assembly. It will be appreciated that an assembly tool can have any suitable configuration and such that the needle guard can be assembled without deploying the needle guard mechanism. It will be appreciated that the assembly tool can be removed after assembly is complete and an end cap can be associated with the needle guard. In an alternate embodiment, the assembly tool and an end cap can be the same component, where the end cap is used to attach the needle guard to a syringe, or the like.

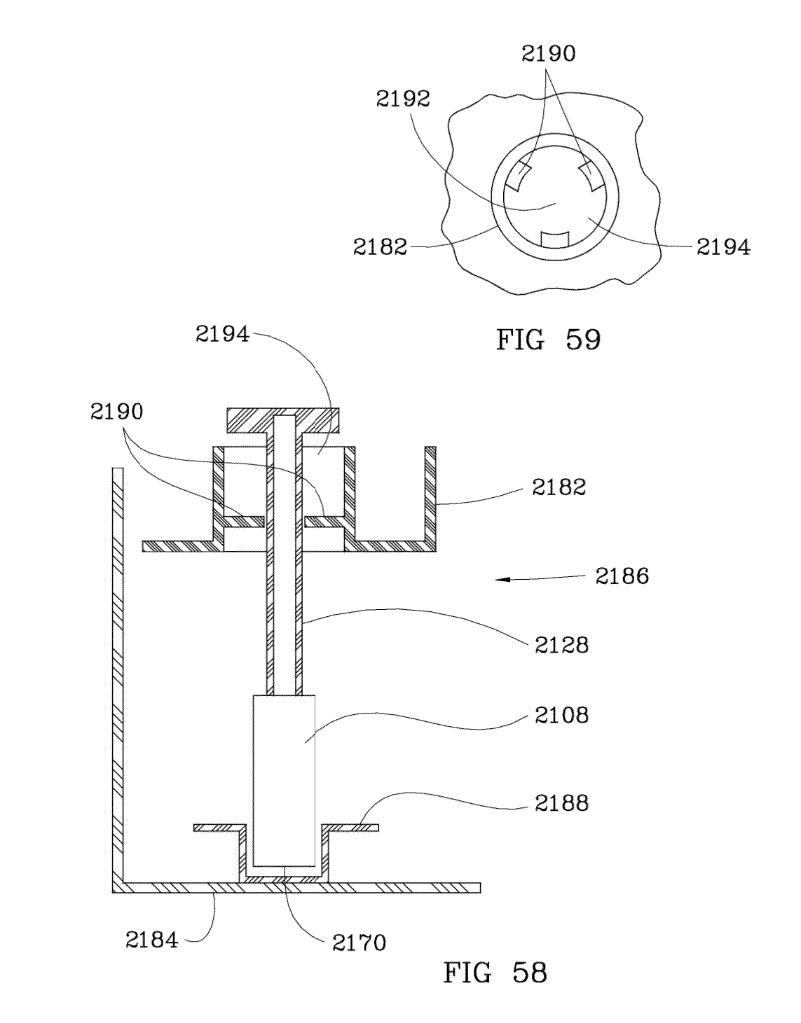

Referring to FIGS. 58 and 59, one embodiment of a nest 2182 and tub 2184 system 2186 can include elements or features configured for the bulk packaging of automatic non-reusable needle guards and standard needle retaining device assemblies, such as those described herein. In one embodiment, the distal end 2170 of a non-reusable needle guard 2108 can be retained in a tray 2188 or other suitable feature or component at the base of the tub 2184 such that movement of the needle guard 2108 is substantially restrained. The nest 2182 can be located proximal to the automatic non-reusable needle guard 2108, and can be in contact or near contact with the needle retaining device 2128. Contact points between the needle retaining device 2128 and the nest 2182 can include flexible elements 2190 that can bias the position of the needle retaining device 2128 at the center of a clearance hole 2192 that can be defined by the flexible elements 2190. The flexible elements 2190 can be manufactured from the same material as the nest 2182, within the nest, or can be composed of differing co-molded, or insert molded materials. The flexible elements 2190 can be separate components that can be bonded or otherwise designed to mechanically remain fixed to the nest 2182. A nest passageway 2194 can be defined by the nest 2182 such that the automatic non-reusable needle guard 2108 and associated needle retaining device 2128 can pass therethrough, where the flexible elements 2190 can be configured to retain the position of the needle guard and needle retaining device while still allowing the components to pass as desired. It will be appreciated that any adjustable aperture or retention system is contemplated that can retain components in a desired position while also allowing the components to pass through during the assembly process. In an alternate embodiment, the flexible arms, members, or flanges, which can be positioned on the nest, can grip the syringe at any suitable location such as a syringe flange or below a finger flange. The arms can be partially flexible, living hinges, rigid structures associated with a hinge, or completely flexible, for example.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art.

We claim:

1. A safety system comprising:
(a) a needle guard assembly, the needle guard assembly being configured for attachment to a needle retaining device having a proximal end, a distal end, and a needle fixedly coupled to the distal end, the needle guard assembly comprising;
(i) a first cylinder, wherein the first cylinder is attached to the distal end of the needle retaining device; and
(ii) a second cylinder, wherein the second cylinder is selectively movable relative to the first cylinder from a first position, to a second position wherein the needle is substantially fully exposed, to a third position wherein the needle is fully covered;
(b) a sterile barrier, wherein the sterile barrier is configured to maintain the sterility of the needle until use; and
(c) a flexible hinge, wherein the flexible hinge is associated with the needle guard assembly and locks the second cylinder in the third position such that the second cylinder cannot be advanced proximally in the third position.

2. The safety system of claim 1, wherein the needle is partially exposed in the first position.

3. The safety system of claim 1, wherein the needle is fully covered in the first position.

4. The safety system of claim 1, wherein the sterile barrier comprises an elastomeric coupling between the distal end of the needle retaining device and the needle guard assembly.

5. The safety system of claim 1, further comprising a removable end cap.

6. The safety system of claim 1, wherein the sterile barrier comprises a tortuous path.

7. The safety system of claim 1, wherein the flexible hinge is in a substantially relaxed state in the first position.

8. A safety system comprising:
(a) a needle guard assembly, the needle guard assembly being configured for attachment to a needle retaining device having a proximal end, a distal end, and a needle fixedly coupled to the distal end, the needle guard assembly comprising a telescoping region having;
(i) a first cylinder, the first cylinder being attached to the distal end of the needle retaining device;
(ii) a second cylinder, the second cylinder being selectively movable relative to the first cylinder; and
(iii) a third cylinder, the third cylinder being selectively movable relative to the second cylinder, wherein the telescoping region includes a first position, a second position wherein the needle is substantially fully exposed, and a third position wherein the needle is fully covered; and
(b) a flexible hinge, wherein the flexible hinge is associated with the needle guard assembly and locks the second cylinder in the third position such that the second cylinder cannot be advanced proximally in the third position.

9. The safety system of claim 8, wherein the needle is partially exposed in the first position.

10. The safety system of claim 8, wherein the first cylinder comprises a first track, the second cylinder comprises a second track and a first projection that engages the first track, and the third cylinder comprises a second projection that engages the second track.

11. The safety system of claim 8, wherein the first cylinder, the second cylinder, and the third cylinder are biased in a distal direction such that the first cylinder, the second cylinder, and the third cylinder move concomitantly.

12. The safety system of claim 8, wherein the first cylinder, the second cylinder, and the third cylinder are biased in a distal direction such that the first cylinder, the second cylinder, and the third cylinder move in series.

13. The safety system of claim 8, wherein the third cylinder is substantially non-rotatable.

14. The safety system of claim 8, further comprising a sterile barrier.

15. The safety system of claim 8, further comprising a spring, wherein the spring is guided such that the spring does not contact the needle during operation of the needle guard assembly.

16. A safety system comprising:
(a) a needle guard assembly, the needle guard assembly being configured for attachment to a needle retaining device having a proximal end, a distal end, and a needle fixedly coupled to the distal end, the needle guard assembly comprising;
(i) a first cylinder, wherein the first cylinder is attached to the distal end of the needle retaining device; and
(ii) a second cylinder, wherein the second cylinder is selectively movable relative to the coupling cylinder from a first position wherein the needle is fully covered, to a second position wherein the needle is substantially fully exposed, to a third position wherein the needle is fully covered; and
(iii) a tension spring, wherein the tension spring couples the second cylinder with the first cylinder; and
(b) a flexible hinge, wherein the flexible hinge is associated with the needle guard assembly and locks the second cylinder in the third position such that the second cylinder cannot be advanced proximally in the third position.

17. A safety system comprising:
(a) a needle guard assembly, the needle guard assembly being configured for attachment to a needle retaining device having a proximal end, a distal end, and a needle fixedly coupled to the distal end, the needle guard assembly comprising;
(i) a first cylinder, wherein the first cylinder is attached to the distal end of the needle retaining device; and
(ii) a second cylinder, wherein the second cylinder is selectively movable relative to the first cylinder from a first position, to a second position wherein the needle is substantially fully exposed, to a third position wherein the needle is fully covered, the second cylinder comprising a first annular portion positioned within the first cylinder and a second annular portion positioned outside of the first cylinder;
(b) a spring, wherein the spring is positioned between the first cylinder and the second annular portion of the second cylinder; and
(c) a flexible hinge, wherein the flexible hinge is associated with the needle guard assembly and locks the second cylinder in the third position such that the second cylinder cannot be advanced proximally in the third position.

18. The safety system of claim 17, wherein the needle is partially covered in the first position.

19. The safety system of claim 17, wherein the needle is fully covered in the first position.

20. The safety system of claim 17, wherein the needle guard assembly is configured to prevent premature deployment during transportation.

21. The safety system of claim 17, where in the needle guard assembly is configured for one step assembly to the needle retaining device.

22. The safety system of claim 17, the first cylinder having a track and the second cylinder having a projection, wherein the projection engages the track such that the track guides the movement of the second cylinder relative to the first cylinder during operation of the needle guard assembly.

23. The safety system of claim 22, wherein the track is substantially J-shaped or G-shaped.

24. The safety system of claim 17, further comprising a pre-use cap to create a sterile barrier for the needle.

25. The safety system of claim 17, wherein the pre-use cap includes a proximally extending arm that engages a sloped stop on the first cylinder such that the stop prevents proximal movement of the second cylinder prior to the pre-use cap being removed.

26. The safety system of claim 17, wherein the second cylinder includes a cylindrical portion that moves proximally over the distal end of the needle retaining device to substantially stabilize the needle guard assembly relative to the needle retaining device.
